(19) United States
(12) Patent Application Publication
(10) Pub. No.: US 2019/0388377 A1
(43) Pub. Date: Dec. 26, 2019

(54) COMPOSITIONS AND METHODS FOR THE TREATMENT OF LIVER DISEASES AND DISORDERS

(71) Applicant: AXCELLA HEALTH INC., Cambridge, MA (US)

(72) Inventors: Michael Hamill, Wellesley, MA (US); Tony Tramontin, Brooklyn, NY (US); Manu Chakravarthy, Newton, MA (US); Raffi Afeyan, Boston, MA (US); Svetlana Marukian, Watertown, MA (US)

(21) Appl. No.: 16/446,412

(22) Filed: Jun. 19, 2019

Related U.S. Application Data
(60) Provisional application No. 62/687,732, filed on Jun. 20, 2018.

(51) Int. Cl. A61K 31/198; A61K 38/05; A61P 1/16

(52) U.S. Cl. CPC A61K 31/198; A61P 1/16; A61K 38/05

(57) ABSTRACT

This disclosure provides compositions and methods for improving liver function, e.g., in a subject having a liver disease or disorder, or treating a liver disease or disorder.

### RELATED APPLICATIONS

This application claims priority to U.S. Ser. No. 62/687,732, filed Jun. 20, 2018, the contents of which is incorporated herein by reference in its entirety.

### BACKGROUND

Non-alcoholic fatty liver disease (NAFLD) is a disease characterized by fatty deposits in the liver due to causes other than alcohol. NAFLD is the most prevalent liver disease in developed countries and affects close to 25% of the people in the United States. Non-alcoholic steatohepatitis (NASH) is the most severe form of NAFLD, which can lead to fibrosis, cirrhosis, chronic liver failure, and hepatocellular carcinoma (HCC).

Currently, there are no approved therapies for treating NASH or NAFLD. Accordingly, there is an unmet need for new treatments for liver diseases and disorders, such as NAFLD and NASH.

### SUMMARY OF THE INVENTION

Provided herein is a composition (e.g., an Active Moiety) including amino acid entities that is useful for improving liver function in a subject, e.g., a subject with a liver disease or disorder. The composition can be used in a method of treating (e.g., reversing, reducing, ameliorating, or preventing) a liver disease or disorder in a subject in need thereof (e.g, a human). The method can further include monitoring the subject for an improvement in one or more symptoms of a liver disease or disorder after administration of the composition.

In one aspect, the invention features a composition comprising:

a) a leucine amino acid entity,

b) a arginine amino acid entity,

c) glutamine amino acid entity;

d) a N-acetylcysteine (NAC) entity; and

e) one or both of a serine amino acid entity or a carnitine entity.

In some embodiments, the total wt. % of (a)-(e) is greater than the total wt. % of other amino acid entities in the composition (e.g., in dry form).

In some embodiments, the composition (e.g., the Active Moiety) further comprises: (f) an isoleucine amino acid entity.

In some embodiments, the composition does not comprise a peptide of more than 20 amino acid residues in length (e.g., whey), or if a peptide of more than 20 amino acid residues in length is present, the peptide is present at less than: 10 weight (wt.) %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, or less, e.g., of the total wt. of the composition (e.g., in dry form).

In some embodiments, one, two, three, four, five, or more (e.g., all) of (a)-(f) are in free amino acid form in the composition, e.g., at least: 42 wt. %, 75 wt. %, 90 wt. %, or more, of the total wt. of the composition (e.g., in dry form) is one, two, three, four, five, or more (e.g., all) of (a)-(f) in free amino acid form in the composition.

In some embodiments, the total wt. % of (a)-(e) or (a)-(f) is greater than the total wt. % of non-amino acid entity protein components (e.g., whey protein) or non-protein components (or both) in the composition (e.g., in dry form), e.g., (a)-(e) or (a)-(f) is at least: 50 wt. 75 wt. %, or 90 wt. % of the total wt. of the total components in the composition (e.g., in dry form).

In some embodiments, the composition comprises a combination of 18 or fewer, 15 or fewer, or 10 or fewer amino acid entities, e.g., the combination comprising at least: 42 wt. %, 75 wt. %, or 90 wt. % of the total wt. of amino acid entity components or total components in the composition (e.g., in dry form).

In some embodiments, the composition does not comprise a peptide of more than 20 amino acid residues in length (e.g., whey protein), or if a peptide of more than 20 amino acid residues in length is present, the peptide is present at less than: 10 wt. %, 1 wt. %, 0.5 wt. %, 0.1 wt. %, 0.05 wt. %, 0.01 wt. %, 0.001 wt. %, or less of the total wt. of the total components of the composition (e.g., in dry form).

In some embodiments, one, two, three, or more (e.g., all) of methionine, tryptophan, valine, or cysteine is absent from the composition, or if present, are present at less than: 10 wt. %, 1 wt. %, 0.5 wt. %, 0.1 wt. %, 0.05 wt. %, 0.01 wt. %, 0.001 wt. %, or less, e.g., of the total wt. of the composition (e.g., in dry form). In some embodiments, one, two, three, or more (e.g., all) of methionine, tryptophan, valine, or cysteine, if present, are present in free form. In some embodiments, one, two, three, or more (e.g., all) of methionine, tryptophan, valine, or cysteine, if present, are present in salt form.

In some embodiments, valine is absent from the composition, or if present, is present at less than: 10 wt. %, 1 wt. %, 0.5 wt. %, 0.1 wt. %, 0.05 wt. %, 0.01 wt. %, 0.001 wt. %, or less, e.g., of the total wt. of the composition (e.g., in dry form).

In some embodiments, methionine, tryptophan, valine, or cysteine, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate.

In some embodiments, at least one, two, three, four, five, or more (e.g., all) of (a)-(f) is selected from Table 1.

In some embodiments, the wt. % of the leucine amino acid entity, the arginine amino acid entity, the glutamine amino acid entity, and the NAC entity is at least: 20 wt. % or 40 wt. % of the amino acid entity components or total components in the composition (e.g., in dry form), but not more than 70 wt. % of the amino acid entity components or total components in the composition (e.g., in dry form).

In some embodiments, the wt. % of the NAC entity is at least: 3 wt. % or 5 wt. % of the amino acid entity components or total components in the composition (e.g., in dry form), but not more than 10 wt. % of the amino acid entity components or total components in the composition (e.g., in dry form).

In some embodiments, the serine amino acid entity is present at a greater wt. % than one, two, or more (e.g., all) of any other amino acid entity, non-amino acid entity protein component, or non-protein component in the composition (e.g., in dry form). In some embodiments, the wt. % of the serine amino acid entity is at least: 20 wt. %, 32 wt. %, or 35 wt. % of the amino acid entity components or total components in the composition (e.g., in dry form), but not more than 70 wt. % of the amino acid entity components or total components in the composition (e.g., in dry form).

In some embodiments, the wt. ratio of the leucine amino acid entity, the arginine amino acid entity, the glutamine amino acid entity, and the NAC entity is 3+/−20%:4+/−20%:2+/−20%:1.3+/−20%. In some embodiments, the wt. ratio of the leucine amino acid entity, the isoleucine amino acid entity, the arginine amino acid entity, the glutamine amino acid entity, the NAC entity, the carnitine entity, and the serine amino acid entity is 3+/−20%:1.5+/−20%:4+/−20%:2+/−20%:1.3+/−20%:0.9+/−20%:7.5+/−20%.

In some embodiments, the composition (e.g., the Active Moiety) comprises:

a) the leucine amino acid entity is chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;

b) the arginine amino acid entity is chosen from:
i) L-arginine or a salt thereof,
ii) a dipeptide or salt thereof, or tripeptide or salt thereof, comprising L-arginine,
iii) creatine or a salt thereof, or
v) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine;

d) the NAC entity is NAC or a salt thereof or a dipeptide or salt thereof, comprising NAC; and

e) one or both of:
i) the serine amino acid entity is L-serine or a salt thereof or a dipeptide or salt thereof, or tripeptide or salt thereof, comprising L-serine; or
b) the carnitine entity is L-carnitine or a salt thereof or a dipeptide or salt thereof, comprising L-carnitine.

In some embodiments, the composition (e.g., the Active Moiety) further comprises: f) L-isoleucine or a salt thereof or a dipeptide or salt thereof, or tripeptide or salt thereof, comprising L-isoleucine.

In some embodiments, the composition (e.g., the Active Moiety) comprises:

a) the leucine amino acid entity is L-leucine or a salt thereof;

b) the arginine amino acid entity is L-arginine or a salt thereof;

c) the glutamine amino acid entity is L-glutamine or a salt thereof;

d) the NAC entity is NAC or a salt thereof;

e) one or both of the serine amino acid entity is L-serine or a salt thereof or the carnitine entity is L-carnitine or a salt thereof; and

f) the isoleucine amino acid entity is L-isoleucine or a salt thereof.

In some embodiments, the composition is present in a unit dosage form comprising 6.7 g+/−20% of amino acid entities.

In some embodiments, the composition (e.g., the Active Moiety) is formulated with a pharmaceutically acceptable carrier.

In some embodiments, the composition (e.g., the Active Moiety) is formulated as a dietary composition. In some embodiments, the dietary composition is chosen from a medical food, a functional food, or a supplement.

In some embodiments, the composition is a dry blended preparation, e.g., pharmaceutical grade dry blended preparation (PGDBP). In another aspect, the invention features a composition for use in a method for improving liver function in a subject in need thereof, comprising an effective amount of the composition of any of the aspects or embodiments disclosed herein.

In another aspect, the invention features a composition for use in a method for treating a symptom chosen from one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress in a subject in need thereof, comprising an effective amount of any of the aspects or embodiments disclosed herein.

In another aspect, the invention features a composition for use in a method for treating a liver disease or disorder in a subject in need thereof, comprising an effective amount of any of the aspects or embodiments disclosed herein.

In another aspect, the invention features a method for improving liver function, comprising administering to a subject in need thereof an effective amount of a composition (e.g., an Active Moiety) of any of the aspects or embodiments disclosed herein, thereby improving liver function in the subject.

In another aspect, the invention features a method for treating a symptom chosen from one, two, three, four, five, six, seven, eight, nine, ten, eleven, or more (e.g., all) of: decreased fat metabolism, hepatocyte apoptosis, hepatocyte ballooning, inflammation of adipose tissue, inflammation of hepatic tissue, fibrosis, liver injury, steatosis, oxidative stress, decreased gut barrier function, decreased insulin secretion, or decreased glucose tolerance, comprising administering to a subject in need thereof an effective amount of a composition (e.g., an Active Moiety) of any of the aspects or embodiments disclosed herein, thereby treating the symptom in the subject.

In another aspect, the invention features a method for treating a liver disease or disorder, comprising administering to a subject in need thereof an effective amount of a composition (e.g., an Active Moiety) of any of the aspects or embodiments disclosed herein, thereby treating the liver disease or disorder in the subject.

In some embodiments, the subject has a fatty liver disease or disorder.

In some embodiments, the fatty liver disease or disorder is chosen from: non-alcoholic fatty liver disease (NAFLD) or alcoholic fatty liver disease (AFLD).

In certain embodiments, the NAFLD is chosen from: non-alcoholic steatohepatitis (NASH) or non-alcoholic fatty liver (NAFL).

In certain embodiments, the subject (e.g., a child or an adolescent) has pediatric NAFLD.

In certain embodiments, the AFLD is alcoholic steatohepatitis (ASH).

In some embodiments, the subject has one, two, three, four, five, or more (e.g., all) of cirrhosis, fibrosis, hepatocarcinoma, steatosis, an increased risk of liver failure, or an increased risk of death.

In some embodiments, the subject has one, two, three, four, five, six, or more (e.g., all) of type 2 diabetes, metabolic syndrome, a high BMI, obesity, gut leakiness, gut dysbiosis, or gut microbiome disturbance.

In some embodiments, administration of the composition results in one, two, three, four, five, six, seven, or more (e.g., all) of: decreasing or preventing liver fibrosis; decreasing or preventing liver injury; decreasing or preventing hepatocyte inflammation; improving glucose tolerance; improving insulin resistance; decreasing or preventing steatosis; decreasing or preventing hepatocyte ballooning; or improving gut function.

In some embodiments, the method further comprises determining the level of one, two, three, four, five, six, seven, eight, nine, 10, 11, 12, 13, 14, 15, 16, 17, 18, 19, 20, 21, 22, or more (e.g., all) of:

a) alanine aminotransferase (ALT); b) aspartate aminotransferase (AST); c) adiponectin;

d) N-terminal fragment of type III collagen (proC3); e) caspase-cleaved keratin 18 fragments (M30 and M65); f) IL-1β; g) C-reactive protein; h) PIIINP; i) a tissue inhibitor of metalloproteinase (TIMP); e.g., TIMP1 or TIMP2; j) MCP-1; k) FGF-21; 1) Col1a1; m) Acta2; n) a matrix metalloproteinase (MMP), e.g., MMP-13, MMP-2, MMP-9, MT1-MMP, MMP-3, or MMP-10; o) ACOX1; p) IL-10; q) NF-kB; r) TNF-α; s) hydroxyproline; t) IL-2; u) MIP-1; v) α-SMA; or w) TGF-β. In another aspect, the invention features a composition for use in a method for treating a diabetic condition in a subject in need thereof (e.g., a subject having diabetic peripheral neuropathy), comprising an effective amount of the composition of any of the aspects or embodiments disclosed herein.

In another aspect, the invention features a method for treating a diabetic condition, comprising administering to a subject in need thereof an effective amount of a composition (e.g., an Active Moiety) of any of the aspects or embodiments disclosed herein, thereby treating the diabetic condition in the subject.

In certain embodiments, the subject has diabetic peripheral neuropathy. In another aspect, the invention features method of manufacturing a dry blended preparation, e.g., PGDBP, comprising at least 4 pharmaceutical grade amino acid entities, said method comprising:

forming a combination of at least 4 pharmaceutical grade amino acid entities and blending the combination for a time sufficient to achieve a dry blended preparation, e.g., PGDBP,

wherein the dry blended preparation, e.g., PGDBP, comprises:
a) a leucine amino acid entity,
b) a arginine amino acid entity,
c) glutamine amino acid entity,
d) a N-acetylcysteine (NAC) entity, and
e) one or both of serine amino acid entity or a carnitine entity.

In certain embodiments, the dry blended preparation, e.g., PGDBP, further comprises (f) an isoleucine amino acid entity.

In another aspect, the invention features a method of manufacturing a dry blended preparation, e.g., PGDBP, comprising at least 4 pharmaceutical grade amino acid entities, said method comprising:

forming a combination of at least 4 pharmaceutical grade amino acid entities and blending the combination for a time sufficient to achieve a dry blended preparation, e.g., PGDBP,

wherein the dry blended preparation, e.g., PGDBP, comprises:
a) a leucine amino acid entity,
b) an isoleucine amino acid entity,
c) a arginine amino acid entity,
d) a N-acetylcysteine (NAC) entity; and
e) a carnitine entity.

In some embodiments, the dry blended preparation, e.g., PGDBP, further comprises (f) one or both of a glutamine amino acid entity or a serine amino acid entity.

In certain embodiments, one, two, or three of:

(i) blending occurs at a temperature lower than 40° C.;

(ii) blending comprises blending or mixing in a blender or mixer at a speed of less than 1000 rpm; or

(iii) the method further comprises performing one, two, or three of direct blending, roller compaction, or wet granulation on the dry blended preparation, e.g., PGDBP.

In another aspect, the invention features a composition comprising:

a) a leucine amino acid entity,

b) an isoleucine amino acid entity,

c) a arginine amino acid entity,

d) a N-acetylcysteine (NAC) entity; and

e) a carnitine entity;

wherein the total wt. % of (a)-(e) is greater than the total wt. % of other amino acid entities in the composition (e.g., in dry form); and

wherein optionally the wt. % of the carnitine entity is at least 2 wt. % of the amino acid entity components or total components in the composition.

In some embodiments, the composition further comprises: (f) one or both of a glutamine amino acid entity or a serine amino acid entity.

In some embodiments, the wt. ratio of the leucine amino acid entity, the isoleucine amino acid entity, the arginine amino acid entity, the NAC entity, and the carnitine entity is 3+/−20%:1.5+/−20%:4+/−20%:1.3+/−20%:0.9+/−20%.

In some embodiments, the wt. ratio of the leucine amino acid entity, the isoleucine amino acid entity, the arginine amino acid entity, the NAC entity, and the carnitine entity is 3+/−20%:1.5+/−20%:4.5+/−20%:1.3+/−20%:1.0+/−20%.

In some embodiments, the composition comprises:

a) the leucine amino acid entity is chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;

b) the isoleucine amino acid entity is L-isoleucine or a salt thereof, or a dipeptide or salt thereof, or tripeptide or salt thereof, comprising L-isoleucine;

c) the arginine amino acid entity is chosen from:
i) L-arginine or a salt thereof,
ii) a dipeptide or salt thereof, or tripeptide or salt thereof, comprising L-arginine,
iii) creatine or a salt thereof, or
v) a dipeptide or salt thereof, or tripeptide or salt thereof, comprising creatine;

d) the NAC entity is NAC or a salt thereof or a dipeptide or salt thereof, comprising NAC; and

e) the carnitine entity is L-carnitine or a salt thereof or a dipeptide or salt thereof, or tripeptide or salt thereof, comprising L-carnitine.

In some embodiments, the composition further comprises: f) the glutamine amino acid entity is L-glutamine or a salt thereof or a dipeptide or salt thereof, or tripeptide or salt thereof, comprising L-glutamine; or the serine amino acid entity is L-serine or a salt thereof or a dipeptide or salt thereof, or tripeptide or salt thereof, comprising L-serine.

In some embodiments, the composition comprises:

a) the leucine amino acid entity is L-leucine or a salt thereof;

b) the isoleucine amino acid entity is L-isoleucine or a salt thereof;

c) the arginine amino acid entity is L-arginine or a salt thereof;

d) the NAC entity is NAC or a salt thereof;

e) the carnitine entity is L-carnitine or a salt thereof; and

f) one or both of the glutamine amino acid entity is L-glutamine or a salt thereof or the serine amino acid entity is L-serine or a salt thereof.

### BRIEF DESCRIPTION OF THE DRAWING

FIGS. 1A-1L are images showing lipid accumulation in primary human hepatocytes from healthy donors (FIG. 1A-1C), treated with free fatty acids (FF) and TNFα (FIG. 1D-1F), treated with LIRNAC (FIG. 1G-1I), or treated with LIRNAC and L-carnitine (FIG. 1J-1L).

FIG. 2A shows alignments of NIR spectrographs taken at increasing blending times (0, 5, 10, 15, 20, 25, 30, and 35 minutes) of a PGDBP.

FIG. 2B is a graph showing the average amount and standard error of amino acid in 10 random samples from the 25 minute blending time of a PGDBP (the PGDBP of FIG. 2A).

### DETAILED DESCRIPTION

Described herein, in part, is a composition (e.g., an Active Moiety) comprising amino acid entities and methods of improving liver function by administering an effective amount of the composition. The composition may be administered to treat or prevent a liver disease or disorder in a subject in need thereof. The amino acid entities and relative amounts of the amino acid entities in the composition have been carefully selected, e.g., to improve liver function in a subject (e.g., a subject having a liver disease or disorder) that requires the coordination of many biological, cellular, and molecular processes. The composition allows for multi-pathway beneficial effects on liver function to optimize modulation of signaling pathways involved in inflammation, lipid and glucose metabolism, and hepatic fat accumulation and mitochondrial function. In particular, the composition has been specifically tailored to improve insulin sensitivity, reduce steatosis, inflammation and fibrosis, and increase fatty acid oxidation.

NAFLD is a multifactorial disorder driven by inflammation, insulin resistance, lipotoxicity, and fibrosis. NAFLD encompasses a histological spectrum, ranging from steatosis, non-alcoholic steatohepatitis (NASH), fibrosis, cirrhosis, and carries a risk of progression to hepatocellular carcinoma. The global prevalence of NAFLD is currently estimated to be 24%. The prevalence of NAFLD has increased substantially in the past two decades and has become the most common cause of chronic liver disease in the pediatric population. Despite the increasing prevalence and progressive nature of disease, therapy for NAFLD in both adults and children is limited. The current standard of care for patients with adult and pediatric NAFLD is lifestyle modification with no approved pharmacological interventions.

The compositions disclosed herein are designed to address the multifactorial nature of NAFLD by restoring insulin sensitivity, reducing oxidative stress, increasing fatty acid oxidation and mitochondrial function, improving the intestinal barrier function, and reducing inflammation and fibrosis.

The compositions of the invention can reprogram the multifactorial biology of hepatocytes, macrophages and hepatic stellate cells (HSC), and are designed to target multiple pathways (metabolism, inflammation and fibrosis) and affect key organ systems (liver, muscle, adipose tissue, gut) to maintain liver health and function.

The amino acid entities and their relative ratios in the compositions disclosed herein have been optimized to a) modulate metabolism by lowering lipotoxicity, improving insulin sensitivity and increasing fatty acid oxidation, b) reduce inflammation by reprogramming macrophages towards less inflammatory phenotypes, reducing hepatic inflammation, and improving gut integrity, c) reduce hepatic fibrogenesis by reducing HSC activation, proliferation and collagen production.

Complex diseases, such as liver diseases or disorders, impact multiple biological pathways. Loss of health can be the direct result of metabolic pathways and functions that are not being maintained or supported. Consequently, restoring homeostasis and maintaining health requires multifactorial approaches. The compositions described herein are interventional candidates to address the systems-wide impact of dysregulated metabolism to support and maintain homeostasis, which helps support normal structures and functions of the body.

The composition described herein have been optimized to directly and simultaneously target multiple metabolic pathways implicated both in complex diseases (e.g., a subject having a liver disease or disorder) and overall health. The distinct ratios of each of the amino acid entities in the composition are designed to target multiple pathways including metabolism (e.g., one, two, or three of lowering lipotoxicity, improving insulin sensitivity, or maximizing mitochondrial function by enhancing fatty acid beta-oxidation), inflammation (e.g., one, two, or three of modulating macrophage function, reducing hepatic inflammatory mediators, or improving gut epithelial integrity), and fibrosis (e.g., one or both of reducing hepatic stellate cell activation or proliferation to decrease hepatic fibrogenesis). In particular, the composition described herein can support and maintain liver health, which is critical to a multitude of metabolic functions throughout the body. In some embodiments the composition described herein activates signaling pathways for protein synthesis; avoids potential accumulation of 3-hydroxybutyrate (3HB); optimizes exposure of a glutamine amino acid entity in gut; minimizes systemic exposure of a glutamine amino acid entity; decreases inflammation; reduces fibrosis; increases fat metabolism shuttling fatty acids into mitochondria for oxidation; and/or decreases metabolic dysregulation of Serine/Glycine biology, e.g., in a subject (e.g., a subject with a liver disease or disorder).

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form.

Salts of amino acids include any ingestible salt. For pharmaceutical compositions, the salt form of an amino acid present in the composition (e.g., Active Moiety) should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino add entities include amino acids, precursors, metabolites,
and derivatives of the compositions described herein.

| | Exemplary | | | |
|---|---|---|---|---|
| | Amino Acid | Precursors | Metabolites | Derivatives |
| Leucine | L-Leucine | Oxo-leucine | HMB (beta- | N-Acetyl- |
| | | | hydroxy-beta- | Leucine |
| | | | methybutyrate); | |
| | | | Oxo-leucine; | |
| | | | Isovaleryl-CoA | |
| Isoleucine | L-Isoleucine | 2-Oxo-3-methyl- | 2-Oxo-3-methyl- | N-Acetyl- |
| | | valerate | valerate; | Isoleucine |
| | | | Methylbutyrl-CoA | |
| Arginine | L-Arginine | Argininosuccinate; | Agmatine; | N-Acetyl- |
| | | Aspartate; Glutamate | Creatine | Arginine |
| Glutamine | L-Glutamine | Glutamate | Carbamoyl-P; | N-Acetyl- |
| | | | Glutamate | Glutamine; |
| NAC | N-Acetylcysteine | Acetylserine; | Glutathione; | Cystine; |
| | | Cystathionine | Cystathionine; | Cysteamine |
| | | | Homocysteine; | |
| | | | Methionine | |
| Serine | L-Serine | Phosphoserine, P- | Glycine, | |
| | | hydroxypyruvate, L- | Tryptophan, | |
| | | Glycine | Acetylserine, | |
| | | | Cystathionine, | |
| | | | Phosphatidylserine | |
| Carnitine | L-Carnitine | 6-N-trimethyllysine; | | Acetyl-L- |
| | | N6-Trimethyl-3-OH- | | Carnitine |
| | | lysine | | (ALCAR); |
| | | | | Proprionyl-L- |
| | | | | Carnitine |
| | | | | (PLCAR); L- |
| | | | | Carnitine L- |
| | | | | Tartrate |

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids.

As used herein, the term “Active Moiety” means a combination of four or more amino acid entities that, in aggregate, have the ability to have a physiological effect as described herein, e.g., improving liver function. For example, an Active Moiety can rebalance a metabolic dysfunction in a subject suffering from a disease or disorder. An Active Moiety of the invention can contain other biologically active ingredients. In some examples, the Active Moiety comprises a defined combination of four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions”.

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g, a capsule shell, for example), and apportioned into a particular dose (e.g., in multiple stick packs).

As used herein, the terms “treat,” “treating,” or “treatment” of a liver disease or disorder refers to ameliorating a liver disease or disorder (e.g., slowing, arresting, or reducing the development of a liver disease or disorder or at least one of the clinical symptoms thereof); alleviating or ameliorating at least one physical parameter including those which may not be discernible by the patient; and/or preventing or delaying the onset or development or progression of a liver disease or disorder.

A “time sufficient” or “sufficient time” as used herein in the context of blending means a time sufficient to achieve blend and composition uniformity without generating impurities or inducing heterogeneity.

A dry blended preparation, e.g., PGDBP, described herein may be formulated as a “pharmaceutical composition”. A pharmaceutical composition as described herein comprises at least one amino acid entity, e.g., an Active Moiety, and a pharmaceutically acceptable carrier or excipient. In some embodiments, the pharmaceutical composition is used as a therapeutic or a medical food. In some embodiments, the pharmaceutical composition is used as a nutriceutical or as a supplement.

The term “pharmaceutical grade” as used herein, refers to amino acids, materials, excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments, pharmaceutical grade means that the amino acids, materials, or excipients meet the specifications of a monograph, e.g., a monograph of the United States Pharmacopeia (USP), the National Formulary (NF), British Pharmacopeia (BP), European Pharmacopeia (EP), or Japanese Pharmacopeia (JP) detailing tests and acceptance criteria. In some embodiments, the meaning of pharmaceutical grade comprises that the amino acids, excipients, or materials are at least 99% pure.

A dry blended preparation, as used herein, means a combination of a plurality of amino acid entities that substantially lacks water. In some embodiments, a dry blended preparation is a powder. In some embodiments, a dry blended preparation comprises less than or equal to 10, 9, 8, 7, 6, 5, 4, 3, 2, or 1% water by weight. In some embodiments, a dry blended preparation comprises at least 4 amino acid entities, e.g., 4, 5, 6, 7, 8, 9, 10, 11, 12, 13, 14, 15, 16, 17, 18, 19, or 20 amino acid entities.

A pharmaceutical grade dry blended preparation (PGDBP), as used herein, is a dry blended preparation that meets a reference standard (e.g., one or more reference standards) and comprises a plurality of pharmaceutical grade amino acid entities. A PGDBP may be formulated as a pharmaceutical composition, e.g., the PGDBP may further comprise one or more excipients and/or oral administration components. In some embodiments, a reference standard met by a PGDBP is composition uniformity.

Composition uniformity, as used herein, is a standard for the homogeneity of a component of a combination, e.g., a dry blended preparation, e.g., a PGDBP, that comprises blend uniformity, portion uniformity, or both. In some embodiments, a combination meets a standard for composition uniformity, e.g., blend uniformity, if the amount of a component (e.g., a pharmaceutical grade amino acid entity, excipient, or oral administration component) at a sampling point in the combination differs from a reference value by less than a predetermined amount. In some embodiments, the reference value is the amount of the component at a second sampling point in the combination. In some embodiments, the reference value is the amount of the component (e.g., a pharmaceutical grade amino acid entity, excipient, or oral administration component) present in the combination (e.g., a dry blended preparation, e.g., a PGDBP).

In some embodiments, wherein a combination (e.g., a dry blended preparation, e.g., a PGDBP) is divided into portions, the portions of the combination meet a standard for composition uniformity, e.g., portion uniformity, if the amount of a component (e.g., a pharmaceutical grade amino acid entity, excipient, or oral administration component) in a portion differs from a reference value by less than a predetermined amount. In some embodiments, the reference value is the amount of the component in a second portion. In some embodiments, the reference value comprises the amount of the component in a N additional portions, wherein it is at least 2, 3, 4, 5, 6, 7, 8, 9, 10, 20, 30, 40, 50, 60, 70, 80, 90, or 100. In some embodiments, the reference value is the amount of the component (e.g., a pharmaceutical grade amino acid entity, excipient, or oral administration component) present in the combination (e.g., a dry blended preparation, e.g., a PGDBP). Amounts may be absolute (e.g., mass or weight) or relative (e.g., percent of total components). In some embodiments, the predetermined amount may be 20, 19, 18, 17, 16, 15, 14, 13, 12, 11, 10, 9, 8, 7, 6, 5, 4, 3, 2, or 1%, e.g., of the reference value. In some embodiments, the predetermined amount is 10% (e.g., the amount of the component differs from the reference value by less than 10%).

Portioning, as used herein, means dividing all or part of the dry blended preparation, e.g., PGDBP, into portions for administration to a patient or subject. The portions created by portioning may be provided in sachets, vials, or other containers, e.g., stick packs. In one embodiment, the portions created by portioning are unit dosage amounts, e.g., one unit dosage or a fraction of a unit dosage (e.g., a stick pack may comprise half a unit dose, such that two stick packs would be used together to provide a single unit dose). In some embodiments, only PGDBPs (e.g., that meet a reference standard) are separated into portions via portioning. In some embodiments, portions generated by portioning also meet a reference standard.

### Compositions Comprising Amino Acid Entities (e.g., Active Moieties)

The composition of the invention as described herein (e.g., an Active Moiety) comprises amino acid entities, e.g., the amino acid entities shown in Table 1. In some embodiments, the composition comprises: a) a leucine amino acid entity, b) a arginine amino acid entity, c) glutamine amino acid entity; d) a N-acetylcysteine (NAC) entity; and e) a serine amino acid entity. In some embodiments, the composition comprises: a) a leucine amino acid entity, b) a arginine amino acid entity, c) glutamine amino acid entity; d) a NAC entity; and e) a carnitine entity. In some embodiments, the composition comprises: a) a leucine amino acid entity, b) a arginine amino acid entity, c) glutamine amino acid entity; d) a NAC entity; and e) a serine amino acid entity and a carnitine entity.

In certain embodiments, the leucine amino acid entity is chosen from L-leucine, β-hydroxy-β-methylbutyrate (HMB), oxo-leucine (α-ketoisocaproate (KIC)), isovaleryl-CoA, n-acetyl-leucine, or a combination thereof.

In certain embodiments, the isoleucine amino acid entity is chosen from L-isoleucine, 2-oxo-3-methyl-valerate (α-keto-beta-methylvaleric acid (KMV)), methylbutyrl-CoA, N-acetyl-isoleucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, glutamate, n-acetylglutamine, or a combination thereof.

In certain embodiments, the NAC entity is selected chosen from NACacetylserine, cystathionine, cystathionine, homocysteine, glutathione, or a combination thereof.

In certain embodiments, the serine amino acid entity is chosen from L-serine, phosphoserine, p-hydroxypyruvate, glycine, acetylserine, cystathionine, phosphatidylserine, or a combination thereof. In some embodiments, the serine amino acid entity is chosen from L-serine or L-glycine. In one embodiment, the serine amino acid entity is L-serine. In another embodiment, the serine amino acid entity is L-glycine. In another embodiment, the serine amino acid entity is L-glycine and L-serine (e.g., L-glycine and L-serine at a wt. ratio of 1:1).

In certain embodiments, the carnitine entity is chosen from L-carnitine, 6-N-trimethyllysine, N6-trimethyl-3-OH-lysine, acetyl-L-carnitine, proprionyl-L-carnitine, L-carnitine L-tartrate, or a combination thereof.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), and a NAC-entity.

In some embodiments, one, two, three, four, five, or more (e.g., all) of (a)-(f) are in free amino acid form in the composition, e.g., at least: 42 wt. %, 75 wt. %, or 90 wt. % of the total wt. of amino acid entity components or total components is one, two, three, four, five, or more (e.g., all) of (a)-(f) in free amino acid form in the composition (e.g., in dry form).

In some embodiments, one, two, three, four, five, or more (e.g., all) of (a)-(f) are in salt form in the composition, e.g., at least: 0.01 wt. %, 0.1 wt. %, 0.5 wt. %, 1 wt. %, 5 wt. %, or 10 wt. %, or more of the total wt. of amino acid entity components or total components is one, two, three, four, five, or more (e.g., all) of (a)-(f) in salt form in the composition.

In some embodiments, one, two, three, four, five, or more (e.g., all) of (a)-(f) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, the composition comprises, consists essentially of, or consists of:

a) a leucine amino acid entity,

b) a arginine amino acid entity,

c) glutamine amino acid entity;

d) a NAC entity; and

e) one or both of a serine amino acid entity and a carnitine entity.

In some embodiments, the composition (e.g., the Active Moiety) comprises, consists essentially of, or consists of:

a) an leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;

b) an arginine amino acid entity chosen from:
i) L-arginine or a salt thereof,
ii) a dipeptide or salt thereof, or tripeptide or salt thereof, comprising L-arginine,
iii) creatine or a salt thereof, or
v) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine;

d) the NAC entity is NAC or a salt thereof or a dipeptide or salt thereof, comprising NAC; and

e) one or both of:
i) the serine amino acid entity is L-serine or a salt thereof or a dipeptide or salt thereof, or tripeptide or salt thereof, comprising L-serine; or
b) the carnitine entity is L-carnitine or a salt thereof or a dipeptide or salt thereof, comprising L-carnitine.

In some embodiments, the composition (e.g., an Active Moiety) comprises, consists essentially of, or consists of:

a) the leucine amino acid entity is L-leucine or a salt thereof;

b) the arginine amino acid entity is L-arginine or a salt thereof;

c) the glutamine amino acid entity is L-glutamine or a salt thereof;

d) the NAC entity is NAC or a salt thereof;

e) one or both of: i) the serine amino acid entity is L-serine or a salt thereof, or ii) the carnitine entity is L-carnitine or a salt thereof; and

f) the isoleucine amino acid entity is L-isoleucine or a salt thereof.

In some embodiments, the composition is capable of one, two, three, four, five, six, seven, or more (e.g., all) of:

a) decreasing or preventing liver fibrosis;

b) decreasing or preventing liver injury;

c) decreasing or preventing hepatocyte inflammation;

d) improving, e.g., increasing, glucose tolerance;

e) decreasing or preventing steatosis;

f) decreasing or preventing hepatocyte ballooning;

g) increasing liver fatty acid oxidation; or

h) improving gut function.

In some embodiments, the composition decreases or prevents one or both of liver fibrosis or liver injury. In some embodiments, the decreasing or preventing one or both of liver fibrosis or liver injury includes reducing a level of collagen, e.g., one or both of type I or III collagen.

In some embodiments, the decreasing or preventing one or both of liver fibrosis or liver injury includes reducing a level or activity of one, two, three, four, five, six, seven, eight, nine, ten, or more (e.g., all) of Acta2; Col1a1; FGF-21; hydroxyproline; IL-1β; a MMP (e.g., MMP-13, MMP-2, MMP-9, MT1-MMP, MMP-3, or MMP-10); proC3; PIINP; αSMA; TGFβ; or TIMP (e.g., TIMP1 or TIMP2).

In some embodiments, the composition decreases or prevents liver inflammation (e.g., hepatocyte inflammation). In some embodiments, the decreasing or preventing liver inflammation includes reducing a level or activity of one, two, three, four, five, six, seven, eight, nine, or more (e.g., all) of aspartate transaminase (AST); alanine transaminase (ALT); C-reactive protein; IL-1β; IL-2; MCP-1; MIP-1; NF-kB; or TNFα. In some embodiments, the decreasing or preventing liver inflammation includes increasing a level or activity of IL-10.

In some embodiments, the composition decreases insulin resistance or increases glucose tolerance. In some embodiments, the decreasing insulin resistance or increasing glucose tolerance includes reducing a level or activity of one, two, three, four, five, or more (e.g., all) of ACOX1; caspase-cleaved keratin 18 fragments (e.g., M30 or M65); FGF-21; hydroxyproline content; IL-1β; or IL-2. In some embodiments, the decreasing insulin resistance or increasing glucose tolerance includes increasing a level or activity of adiponectin.

i. Amounts

An exemplary composition can include 1 g of an leucine amino acid entity, 0.5 g of an isoleucine amino acid entity, 1.33 g of an arginine amino acid entity, 0.67 g of a glutamine amino acid entity, 0.43 g of a NAC entity, 0.30 g of a carnitine entity, and 2.5 of a serine amino acid entity for a total of 6.73 g+/−20% (e.g., g/packet as shown in Table 2).

TABLE 2
Exemplary composition comprising amino
acids (e.g., an Active Moiety).

| | Packet | Dose | Total | | |
|---|---|---|---|---|---|
| Amino Acid | (g) | (g) | Daily | Wt. Ratio | Wt. % |
| L-leucine | 1.00 | 3.00 | 6.00 | 3.00 | 14.85 |
| L-isoleucine | 0.50 | 1.50 | 3.00 | 1.50 | 7.43 |
| L-arginine | 1.33 | 4.00 | 8.00 | 4.00 | 19.80 |
| L-glutamine | 0.67 | 2.00 | 4.00 | 2.00 | 9.90 |
| NAC | 0.43 | 1.30 | 2.60 | 1.30 | 6.44 |
| L-carnitine | 0.30 | 0.90 | 1.80 | 0.90 | 4.46 |
| L-serine | 2.50 | 7.50 | 15.00 | 7.50 | 37.13 |
| Total amino acids | 6.73 | 20.20 | 40.40 | 20.20 | 100.00 |

In some embodiments, the composition (e.g., the Active Moiety) includes 1 g+/−20% of an leucine amino acid entity, 0.5 g+/−20% of an isoleucine amino acid entity, 1.33 g+/−20% of an arginine amino acid entity, 0.67 g+/−20% of a glutamine amino acid entity, 0.43 g+/−20% of a NAC entity, 0.30 g+/−20% of a carnitine entity, and 2.5 g+/−20% of a serine amino acid entity. In some embodiments, the composition (e.g., the Active Moiety) includes 1 g+/−15% of an leucine amino acid entity, 0.5 g+/−15% of an isoleucine amino acid entity, 1.33 g+/−15% of an arginine amino acid entity, 0.67 g+/−15% of a glutamine amino acid entity, 0.43 g+/−15% of a NAC entity, 0.30 g+/−15% of a carnitine entity, and 2.5 g+/−15% of a serine amino acid entity. In some embodiments, the composition (e.g., the Active Moiety) includes 1 g+/−10% of an leucine amino acid entity, 0.5 g+/−10% of an isoleucine amino acid entity, 1.33 g+/−10% of an arginine amino acid entity, 0.67 g+/−10% of a glutamine amino acid entity, 0.43 g+/−10% of a NAC entity, 0.30 g+/−10% of a carnitine entity, and 2.5 g+/−10% of a serine amino acid entity. In some embodiments, the composition (e.g., the Active Moiety) includes 1 g+/−5% of an leucine amino acid entity, 0.5 g+/−5% of an isoleucine amino acid entity, 1.33 g+/−5% of an arginine amino acid entity, 0.67 g+/−5% of a glutamine amino acid entity, 0.43 g+/−5% of a NAC entity, 0.30 g+/−5% of a carnitine entity, and 2.5 g+/−5% of a serine amino acid entity.

ii. Ratios

An exemplary composition can include a weight (wt.) ratio of the leucine amino acid entity, the arginine amino acid entity, the glutamine amino acid entity, and the NAC entity of 3:4:2:1.3. In some embodiments, the wt. ratio of the leucine amino acid entity, the isoleucine amino acid entity, the arginine amino acid entity, the glutamine amino acid entity, and the NAC entity is 3+/−20%:4+/−20%:2+/−20%:1.3+/−20%. In some embodiments, the wt. ratio of the leucine amino acid entity, the isoleucine amino acid entity, the arginine amino acid entity, the glutamine amino acid entity, and the NAC entity is 3+/−15%:4+/−15%:2+/−15%:1.3+/−15%. In some embodiments, the wt. ratio of the leucine amino acid entity, the isoleucine amino acid entity, the arginine amino acid entity, the glutamine amino acid entity, and the NAC entity is 3+/−10%:4+/−10%:2+/−10%:1.3+/−10%. In some embodiments, the wt. ratio of the leucine amino acid entity, the isoleucine amino acid entity, the arginine amino acid entity, the glutamine amino acid entity, and the NAC entity is 3+/−5%:4+/−5%:2+/−5%:1.3+/−5%.

An exemplary composition can include a wt. ratio of the leucine amino acid entity, the arginine amino acid entity, the glutamine amino acid entity, and the NAC entity of 3:1.5:4:2:1.3. In some embodiments, the wt. ratio of the leucine amino acid entity, the isoleucine amino acid entity, the arginine amino acid entity, the glutamine amino acid entity, and the NAC entity is 3+/−20%:1.5+/−20%:4+/−20%:2+/−20%:1.3+/−20%. In some embodiments, the wt. ratio of the leucine amino acid entity, the isoleucine amino acid entity, the arginine amino acid entity, the glutamine amino acid entity, and the NAC entity is 3+/−15%:1.5+/−15%:4+/−15%:2+/−15%:1.3+/−15%. In some embodiments, the wt. ratio of the leucine amino acid entity, the isoleucine amino acid entity, the arginine amino acid entity, the glutamine amino acid entity, and the NAC entity is 3+/−10%:1.5+/−10%:4+/−10%:2+/−10%:1.3+/−10%. In some embodiments, the wt. ratio of the leucine amino acid entity, the isoleucine amino acid entity, the arginine amino acid entity, the glutamine amino acid entity, and the NAC entity is 3+/−5%:1.5+/−5%:4+/−5%:2+/−5%:1.3+/−5%.

An exemplary composition can include a wt. ratio of the leucine amino acid entity, the isoleucine amino acid entity, the arginine amino acid entity, the glutamine amino acid entity, the NAC entity, and the carnitine entity of 3:1.5:4:2:1.3:0.9. In some embodiments, the composition includes a wt. ratio of the leucine amino acid entity, the isoleucine amino acid entity, the arginine amino acid entity, the glutamine amino acid entity, the NAC entity, and the carnitine entity of 3+/−20%:1.5+/−20%:4+/−20%:2+/−20%:1.3+/−20%:0.9+/−20%. In some embodiments, the composition includes a wt. ratio of the leucine amino acid entity, the isoleucine amino acid entity, the arginine amino acid entity, the glutamine amino acid entity, the NAC entity, and the carnitine entity of 3+/−15%:1.5+/−15%:4+/−15%:2+/−15%:1.3+/−15%:0.9+/−15%. In some embodiments, the composition includes a wt. ratio of the leucine amino acid entity, the isoleucine amino acid entity, the arginine amino acid entity, the glutamine amino acid entity, the NAC entity, and the carnitine entity of 3+/−10%:1.5+/−10%:4+/−10%:2+/−10%:1.3+/−10%:0.9+/−10%. In some embodiments, the composition includes a wt. ratio of the leucine amino acid entity, the isoleucine amino acid entity, the arginine amino acid entity, the glutamine amino acid entity, the NAC entity, and the carnitine entity of 3+/−5%:1.5+/−5%:4+/−5%:2+/−5%:1.3+/−5%:0.9+/−5%.

An exemplary composition can include a wt. ratio of the leucine amino acid entity, the isoleucine amino acid entity, the arginine amino acid entity, the glutamine amino acid entity, the NAC entity, and the serine amino acid entity of 3:1.5:4:2:1.3:7.5. In some embodiments, the composition includes a wt. ratio of the leucine amino acid entity, the isoleucine amino acid entity, the arginine amino acid entity, the glutamine amino acid entity, the NAC entity, and the serine amino acid entity of 3+/−20%:1.5+/−20%:4+/−20%:2+/−20%:1.3+/−20%:7.5+/−20%. In some embodiments, the composition includes a wt. ratio of the leucine amino acid entity, the isoleucine amino acid entity, the arginine amino acid entity, the glutamine amino acid entity, the NAC entity, and the serine amino acid entity of 3+/−15%:1.5+/−15%:4+/−15%:2+/−15%:1.3+/−15%:7.5+/−15%. In some embodiments, the composition includes a wt. ratio of the leucine amino acid entity, the isoleucine amino acid entity, the arginine amino acid entity, the glutamine amino acid entity, the NAC entity, and the serine amino acid entity of 3+/−10%:1.5+/−10%:4+/−10%:2+/−10%:1.3+/−10%:7.5+/−10%. In some embodiments, the composition includes a wt. ratio of the leucine amino acid entity, the isoleucine amino acid entity, the arginine amino acid entity, the glutamine amino acid entity, the NAC entity, and the serine amino acid entity of 3+/−5%:1.5+/−5%:4+/−5%:2+/−5%:1.3+/−5%:7.5+/−5%.

An exemplary composition can include a wt. ratio of the leucine amino acid entity, the isoleucine amino acid entity, the arginine amino acid entity, the glutamine amino acid entity, the NAC entity, the carnitine entity, and the serine amino acid entity of 3:1.5:4:2:1.3:0.9:7.5. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3+/−20%:1.5+/−20%:4+/−20%:2+/−20%:1.3+/−20%:0.9+/−20%:7.5+/−20%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3+/−15%:1.5+/−15%:4+/−15%:2+/−15%:1.3+/−15%:0.9+/−15%:7.5+/−15%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3+/−10%:1.5+/−10%:4+/−10%:2+/−10%:1.3+/−10%:0.9+/−10%:7.5+/−10%. In some embodiments, the composition includes a wt. ratio of the leucine amino acid entity, the isoleucine amino acid entity, the arginine amino acid entity, the glutamine amino acid entity, the NAC entity, the carnitine entity, and the serine amino acid entity of 3+/−5%:1.5+/−5%:4+/−5%:2+/−5%:1.3+/−5%:0.9+/−5%:7.5+/−5%.

iii. Relationships of Amino Acid Entities

In some embodiments, the wt. % of the leucine amino acid entity in the composition (e.g., in dry form) is greater than the wt. % of the glutamine amino acid entity, e.g., the wt. % of the leucine amino acid entity in the composition (e.g., in dry form) is at least 15% greater than the wt. % of the glutamine amino acid entity entity, e.g., the wt. % of the leucine amino acid entity is at least 20%, 25%, or 30% greater than the wt. % of the glutamine amino acid entity.

In some embodiments, the wt. % of the leucine amino acid entity in the composition (e.g., in dry form) is greater than the wt. % of the isoleucine amino acid entity, e.g., the wt. % of the leucine amino acid entity in the composition (e.g., in dry form) is at least 25% greater than the wt. % of the isoleucine amino acid entity, e.g., the wt. % of the leucine amino acid entity is at least 30%, 40%, or 50% greater than the wt. % of the isoleucine amino acid entity.

In some embodiments, the wt. % of the leucine amino acid entity and the isoleucine amino acid entity in combination in the composition (e.g., in dry form) is greater than the wt. % of the glutamine amino acid entity in the composition (e.g., in dry form), e.g., the wt. % of the leucine amino acid entity and the isoleucine amino acid entity in combination in the composition (e.g., in dry form) is at least 25% greater than the wt. % of the glutamine amino acid entity, e.g., the wt. % of the leucine amino acid entity and the isoleucine amino acid entity in combination is at least 30%, 40%, or 50% greater than the wt. % of the glutamine amino acid entity.

In some embodiments, the isoleucine amino acid entity and the leucine amino acid entity in combination is at least: 15 wt. %, or 20 wt. % of the amino acid entities in the composition (e.g., in dry form), but not more than: 50 wt. % of the amino acid entities in the composition (e.g., in dry form).

In some embodiments, the wt. % of the leucine amino acid entity and the isoleucine amino acid entity in combination in the composition (e.g., in dry form) is greater than the wt. % of the arginine amino acid entity in the composition (e.g., in dry form), e.g., the wt. % of the leucine amino acid entity and the isoleucine amino acid entity in combination in the composition (e.g., in dry form) is at least 5% greater than the wt. % of the arginine amino acid entity, e.g., the wt. % of the leucine amino acid entity and the isoleucine amino acid entity in combination is at least 6%, 8%, or 10% greater than the wt. % of the arginine amino acid entity.

In some embodiments, the wt. % of the arginine amino acid entity in the composition (e.g., in dry form) is greater than the wt. % of the glutamine amino acid entity, e.g., the wt. % of the arginine amino acid entity in the composition (e.g., in dry form) is at least 25% greater than the wt. % of the glutamine amino acid entity, e.g., the wt. % of the arginine amino acid entity is at least 30%, 40%, or 50% greater than the wt. % of the glutamine amino acid entity.

In some embodiments, the wt. % of the arginine amino acid entity in the composition (e.g., in dry form) is greater than the wt. % of the leucine amino acid entity, e.g., the wt. % of the arginine amino acid entity in the composition (e.g., in dry form) is at least 10% greater than the wt. % of the leucine amino acid entity, e.g., the wt. % of the arginine amino acid entity is at least 15%, 20%, or 25% greater than the wt. % of the leucine amino acid entity.

In some embodiments, the wt. % of the serine amino acid entity in the composition (e.g., in dry form) is greater than the wt. % of the leucine amino acid entity, e.g., the wt. % of the serine amino acid entity in the composition (e.g., in dry form) is at least 30% greater than the wt. % of the leucine amino acid entity, e.g., the wt. % of the serine amino acid entity is at least 45%, 50%, or 60% greater than the wt. % of the leucine amino acid entity.

In some embodiments, the wt. % of the serine amino acid entity in the composition (e.g., in dry form) is greater than the wt. % of the isoleucine amino acid entity, e.g., the wt. % of the serine amino acid entity in the composition (e.g., in dry form) is at least 50% greater than the wt. % of the isoleucine amino acid entity, e.g., the wt. % of the serine amino acid entity is at least 60%, 70%, or 80% greater than the wt. % of the isoleucine amino acid entity.

In some embodiments, the wt. % of the serine amino acid entity in the composition (e.g., in dry form) is greater than the wt. % of the arginine amino acid entity, e.g., the wt. % of the serine amino acid entity in the composition (e.g., in dry form) is at least 20% greater than the wt. % of the arginine amino acid entity, e.g., the wt. % of the serine amino acid entity is at least 35%, 40%, or 45% greater than the wt. % of the arginine amino acid entity.

In some embodiments, the wt. % of the serine amino acid entity in the composition (e.g., in dry form) is greater than the wt. % of the glutamine amino acid entity, e.g., the wt. % of the serine amino acid entity in the composition (e.g., in dry form) is at least 40% greater than the wt. % of the glutamine amino acid entity, e.g., the wt. % of the serine amino acid entity is at least 50%, 60%, or 70% greater than the wt. % of the glutamine amino acid entity.

In some embodiments, the wt. % of the serine amino acid entity in the composition (e.g., in dry form) is greater than the wt. % of the leucine amino acid entity and the isoleucine amino acid entity in combination, e.g., the wt. % of the serine amino acid entity in the composition (e.g., in dry form) is at least 20% greater than the wt. % of the leucine amino acid entity and the isoleucine amino acid entity in combination, e.g., the wt. % of the serine amino acid entity is at least 30%, 35%, or 40% greater than the wt. % of the leucine amino acid entity and the isoleucine amino acid entity in combination.

In some embodiments, the wt. % of the leucine amino acid entity, the arginine amino acid entity, the glutamine amino acid entity, the NAC entity, and the serine entity is at least: 50 wt. % or 75 wt. % of the amino acid entity components or total components in the composition (e.g., in dry form), but not more than 95 wt. % of the amino acid entity components or total components in the composition (e.g., in dry form) in the composition (e.g., in dry form).

In some embodiments, the wt. % of the NAC entity in the composition (e.g., in dry form) is greater than the wt. % of the carnitine entity, e.g., the wt. % of the NAC entity in the composition (e.g., in dry form) is at least 10% greater than the wt. % of the carnitine entity, e.g., the wt. % of NAC entity is at least 15%, 20%, or 30% greater than the wt. % of the carnitine entity.

In some embodiments, the wt. % of the carnitine entity is at least: 2 wt. %, 3 wt. %, or 4 wt. % of the amino acid entity components or total components in the composition (e.g., in dry form), but not more than 15 wt. % of the amino acid entity components or total components in the composition (e.g., in dry form).

In some embodiments, the wt. % of the leucine amino acid entity, the arginine amino acid entity, the glutamine amino acid entity, the NAC entity, and the carnitine entity is at least: 25 wt. %, 40 wt. %, or 50 wt. % of the amino acid entity components or total components in the composition (e.g., in dry form), but not more than 80 wt. % of the amino acid entity components or total components in the composition (e.g., in dry form).

In some embodiments, the wt. % of the leucine amino acid entity, the arginine amino acid entity, the glutamine amino acid entity, the NAC entity, and the serine amino acid entity is at least: 60 wt. %, 70 wt. %, or 80 wt. % of the amino acid entity components or total components in the composition (e.g., in dry form), but not more than 95 wt. % of the amino acid entity components or total components in the composition (e.g., in dry form) in the composition (e.g., in dry form).

In some embodiments, the wt. % of the leucine amino acid entity, the arginine amino acid entity, the glutamine amino acid entity, the NAC entity, the serine amino acid entity, and the carnitine entity is at least: 70 wt. %, 80 wt. %, or 90 wt. % of the amino acid entity components or total components in the composition (e.g., in dry form), but not more than 98 wt. % of the amino acid entity components or total components in the composition (e.g., in dry form) in the composition (e.g., in dry form).

In some embodiments, the wt. % of the glutamine amino acid entity is at least: 5 wt. %, 7 wt. %, or 9 wt. % of the amino acid entity components or total components in the composition (e.g., in dry form), but not more than 20 wt. % of the amino acid entity components or total components in the composition (e.g., in dry form).

iv. Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than: 10 weight (wt.) 5 wt. %, 1 wt. %, 0.1 wt. %, 0.05 wt. %, 0.01 wt. %, of the total wt. of non-amino acid entity protein components or total components in the composition (e.g., in dry form).

In some embodiments, the composition comprises a combination of 3 to 19, 3 to 15, or 3 to 10 different amino acid entities; e.g., the combination comprises at least: 42 wt. %, 75 wt. %, or 90 wt. % of the total wt. % of amino acid entity components or total components in the composition (e.g., in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entity components or total components in the composition (e.g., in dry form).

In some embodiments, at least 50%, 60%, 70%, or more of the total grams of amino acid entity components in the composition (e.g., in dry form) are from one, two, three, four, five, or more (e.g., all) of (a)-(f).

In some embodiments, at least: 50%, 60%, 70%, or more of the calories from amino acid entity components or total components in the composition (e.g., in dry form) are from one, two, three, four, five, or more (e.g., all) of (a)-(f).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, or three of S-allyl cysteine, S-allylmercaptocysteine, or fructosyl-arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Uses, e.g., Methods of Treatment

The disclosure provides a method for improving liver function, comprising administering to a subject in need thereof an effective amount of a composition disclosed herein (e.g., an Active Moiety). The composition can be administered according to a dosage regimen described herein to improve liver function in a subject (e.g., a human).

The disclosure provides a method for treating or preventing a liver disease or disorder in a subject, comprising administering to a subject in need thereof an effective amount of a composition disclosed herein (e.g., an Active Moiety). The composition can be administered according to a dosage regimen described herein to treat a liver disease or disorder in a subject (e.g. a human).

In some embodiments, the subject has been diagnosed with a liver disease or disorder. In some embodiments, the subject has not been diagnosed with a liver disease or disorder. In some embodiments, the subject is a human. In some embodiments, the subject has not received prior treatment with the composition described herein (e.g., a naïve subject).

In some embodiments, the composition described herein (e.g., the Active Moiety) is for use as a medicament in improving liver function in a subject (e.g., a subject with a liver disease or disorder). In some embodiments, the composition is for use as a medicament in treating (e.g., reversing, reducing, ameliorating, or preventing) a liver disease or disorder in a subject.

In some embodiments, the composition described herein (e.g., the Active Moiety) is for use in the manufacture of a medicament, supplement, medical food, or functional food for improving liver function in a subject (e.g., a subject with a liver disease or disorder). In some embodiments, the composition (e.g., the Active Moiety) is for use in the manufacture of a medicament, supplement, medical food, or functional food for treating (e.g., reversing, reducing, ameliorating, or preventing) a liver disease or disorder in a subject.

In some embodiments, the composition described herein (e.g., the Active Moiety) is for use in the manufacture of a medicament, supplement, medical food, or functional food for decreasing one, two, three, four, five, six, seven, eight, nine, ten, or more (e.g., all) of: decreased fat metabolism, hepatocyte apoptosis, hepatocyte ballooning, inflammation of adipose tissue, inflammation of hepatic tissue, fibrosis, liver injury, steatosis, glucose tolerance, insulin resistance, or oxidative stress in a subject in need thereof. A subject that may be treated with the composition described herein (e.g., the Active Moiety) includes a subject having a fatty liver disease or disorder. In some embodiments, the fatty liver disease or disorder is chosen from: non-alcoholic fatty liver disease (NAFLD) or alcoholic fatty liver disease (AFLD).

In certain embodiments, the NAFLD is chosen from: non-alcoholic steatohepatitis (NASH) or non-alcoholic fatty liver (NAFL). In certain embodiments, the subject (e.g., a child or an adolescent) has pediatric NAFLD. In certain embodiments, the AFLD is alcoholic steatohepatitis (ASH).

In some embodiments, the subject has one or both of fibrosis or steatosis.

In certain embodiments, the subject (e.g., a subject with NASH) has cirrhosis. In some embodiments, the subject has hepatocarcinoma. In certain embodiments, the subject has one or both of an increased risk of liver failure or an increased risk of death.

In some embodiments, the subject has one, two, three, or more (e.g., all) of diabetes (e.g., type 2 diabetes), metabolic syndrome, a relatively high BMI, or obesity.

In some embodiments, the subject has one, two, or more (e.g., all) of gut leakiness, gut dysbiosis, or gut microbiome disturbance.

In certain embodiments, the subject exhibits muscle atrophy, e.g., has a decreased ratio of muscle tissue to adipose tissue, e.g., relative to a normal subject without a fatty liver disease. For example, the subject exhibits muscle atrophy without fibrosis and/or cirrhosis.

In some embodiments, the subject exhibits a symptom of a liver disease or disorder, e.g., a metabolic symptom. In some embodiments, a subject exhibits a metabolic symptom of liver disease chosen from one, two, three, four, five, six, seven, eight, nine, ten, eleven, or more (e.g., all) of: decreased fat metabolism, hepatocyte apoptosis, hepatocyte ballooning, inflammation of adipose tissue, inflammation of hepatic tissue, fibrosis, liver injury, steatosis, oxidative stress (e.g., one, two, or more (e.g., all) of increased levels of reactive oxygen species (ROS), decreased mitochondrial function, or decreased levels of glutathionine (GSH)), decreased gut barrier function, decreased insulin secretion, or decreased glucose tolerance (e.g., relative to a healthy subject without a liver disease).

In some embodiments, administration of the composition results in an improvement in a metabolic symptom of liver disease in a subject chosen from one, two, three, four, five, six, seven, eight, nine, ten, eleven, twelve, thirteen, fourteen, fifteen, or more (e.g., all) of: increased free fatty acid and lipid metabolism (e.g., in the liver); white adipose tissue (WAT) browning; a reduction in liver fat; decreased hepatocyte apoptosis; decreased hepatocyte ballooning; decreased inflammation of adipose tissue; decreased inflammation of hepatic tissue; a reduction or inhibition of fibrosis; healing of liver injury; decreased steatosis; decreased reactive oxygen species (ROS); improved mitochondrial function; increased levels of glutathione (GSH); improved gut barrier function; increased insulin secretion; or improved glucose tolerance.

In some embodiments, administration of the composition described herein (e.g., the Active Moiety) to a subject reduces the level or activity of a pro-inflammatory cytokine (e.g., one, two, three, or more (e.g., all) of TNFα, IL-1, IL-6, or IFNγ), e.g., relative to a normal subject without a fatty liver disease. In some embodiments, administration of the composition described herein to a subject reduces the level or activity of a pro-inflammatory mediator (e.g., NF-kB), e.g., relative to a normal subject without a fatty liver disease. In some embodiments, administration of the composition described herein to a subject increases the level or activity of a anti-inflammatory cytokine (e.g., one, two, three, or more (e.g., all) of IL-10, IL-4, IL-13, and IL-5), e.g., relative to a normal subject without a fatty liver disease.

In some embodiments, administration of the composition reduces liver enzyme levels (e.g., one or both of ALT or AST) in one or both of blood or plasma from a subject (e.g., a subject with fatty liver disease), e.g., relative to the subject prior to administration of the composition.

The disclosure provides a method for treating a subject with a diabetic condition, comprising administering to a subject in need thereof an effective amount of a composition disclosed herein (e.g., an Active Moiety), thereby treating the subject. In some embodiments, the subject has diabetic peripheral neuropathy.

In certain embodiments, the subject exhibits a symptom of a diabetic condition (e.g., diabetic peripheral neuropathy) chosen from one, two, three, four, five, six, seven, eight, nine, ten, eleven, twelve, thirteen, fourteen, or more (e.g., all) of: trouble with balance; numbness of extremities; tingling of extremities; dysesthesia; diarrhea; erectile dysfunction; loss of bladder control; facial, mouth, or eyelid drooping; vision change; dizziness; muscle weakness; difficulty swallowing; speech impairment; fasciculation; or burning or electric pain.

In certain embodiments, administration of the composition to the subject results in an improvement in a symptom of a diabetic condition (e.g., diabetic peripheral neuropathy) chosen from one, two, three, four, five, six, seven, eight, nine, ten, eleven, twelve, thirteen, fourteen, or more (e.g., all) trouble with balance; numbness of extremities; tingling of extremities; dysesthesia; diarrhea; erectile dysfunction; loss of bladder control; facial, mouth, or eyelid drooping; vision change; dizziness; muscle weakness; difficulty swallowing; speech impairment; fasciculation; or burning or electric pain.

### Dosage Regimens

The composition can be administered according to a dosage regimen described herein to improve liver function in a subject, e.g., to reduce or treat a liver disease or disorder. For example, the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of 2 g+/−20% g daily to 80 g+/−20% g daily (e.g., 30 g+/−20%, 32 g+/−20%, 34 g+/−20%, 38 g+/−20%, 40 g+/−20%, 42 g+/−20%, 44 g+/−20%, 48 g+/−20%, 50 g+/−20%, 52 g+/−20%, 54 g+/−20%, 56 g+/−20%, 58 g+/−20%, or 60 g+/−20% total amino acid entities daily). In certain embodiments, the composition is administered at a dose of 30 g+/−20% to 60 g+/−20% total amino acid entities three times daily, e.g., 30 g+/−20%, 32 g+/−20%, 34 g+/−20%, 38 g+/−20%, 40 g+/−20%, 42 g+/−20%, 44 g+/−20%, 48 g+/−20%, 50 g+/−20%, 52 g+/−20%, 54 g+/−20%, 56 g+/−20%, 58 g+/−20%, or 60 g+/−20% total amino acid entities daily.

In some embodiments, the composition can be provided to a subject with a liver disease or disorder in either a single or multiple dosage regimen. In some embodiments, a dose is administered twice daily, three times daily, four times daily, five times daily, six times daily, seven times daily, or more. In certain embodiments, the composition is administered one, two, or three times daily. In some embodiments, the composition is administered for at least 2 days, 3 days, 4 days, 5 days, 6 days, 7 days, or 2 weeks. In some embodiments, the composition is administered chronically (e.g., more than 30 days, e.g., 31 days, 40 days, 50 days, 60 days, 3 months, 6 months. 9 months, one year, two years, or three years).

In some embodiments, the composition is administered prior to a meal. In other embodiments, the composition is administered concurrent with a meal. In other embodiments, the composition is administered following a meal.

The composition can be administered every 2 hours, every 3 hours, every 4 hours, every 5 hours, every 6 hours, every 7 hours, every 8 hours, every 9 hours, or every 10 hours to improve liver function in a subject (e.g., a subject having a liver disease or disorder).

In some embodiments, the composition comprises three stick packs, e.g., each stick pack comprising 33.3%+/−15% of the quantity of each amino acid entity included in the composition described herein. In certain embodiments, three stick packs are administered two times daily.

In some embodiments, the composition is administered at a dose of about 2 g+/−20% to 60 g+/−20% total amino acid entities, e.g., once daily, twice daily, three times daily, four times daily, five times daily, or six times daily (e.g., twice daily). In some embodiments, the composition is administered at a dose of 2 g+/−20% to 10 g+/−20%, 10 g+/−20% to 30 g+/−20%, or 30 g+/−20% to 60 g+/−20% total amino acid entities, e.g., once daily, twice daily, or three times daily (e.g., twice per day). In certain embodiments, the composition is administered at a dose of 10 g+/−20% to 30 g+/−20% total amino acid entities twice daily, e.g., 10 g+/−20%, 12 g+/−20%, 14 g+/−20%, 16 g+/−20%, 18 g+/−20%, 20 g+/−20%, 22 g+/−20%, 24 g+/−20%, 26 g+/−20%, 28 g+/−20%, 30 g+/−20%, 32 g+/−20%, 34 g+/−20%, 36 g+/−20%, 38 g+/−20%, or 40 g+/−20% total amino acid entities twice daily. In some embodiments, the composition is present in a unit dosage form of 2 g+/−20% to 15 g+/−20% (e.g., 2 g+/−20%, 3 g+/−20%, 4 g+/−20%, 5 g+/−20%, 6 g+/−20%, 7 g+/−20%, 8 g+/−20%, 9 g+/−20%, 10 g+/−20%, 11 g+/−20%, 12 g+/−20%, 13 g+/−20%, 14 g+/−20%, or 15 g+/−20%). In certain embodiments, the composition is present in a unit dosage form of 6.73 g+/−20%.

### Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization. The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.15, 0.1, 0.05, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6). Exemplary ingredient contents for each stick pack are shown in Table 3.

TABLE 3
Ingredient contents in each stick pack.

| INGREDIENT | GRADE | FUNCTION | SOURCE; COMMENT |
|---|---|---|---|
| Amino Acids | USP | Active Pharmaceutical | Various sources; Non- |
| | | Ingredient (API) | instantized form (MFG |
| | | | scale) |
| Citric Acid | USP | pH, Flavor | Spectrum Chems; |
| | | | f(volume) ≤ 1.0% w/v |
| Acesulfame K | NF | Sweetness (rapid onset) | Spectrum Chems; Target 1 |
| | | | Sweetener |
| Sucralose | NF | Sweetness (slow onset) | Spectrum Chems; WHO |
| | | | ADI ≤ 15 mg/kg |
| Lecithin (Alecolec | FCC | Wetting Agent | American Lecithin |
| F100) | | | Company |
| Xanthan Gum | FCC | Stabilizer/Thickener | TIC Gums; f(volume) ≤ |
| | | | 0.5% w/v |
| Vanilla Custard (Art) | GRAS | Taste/Aroma | David Michael; Mask |
| | | | sulfur |
| Orange (Natural and | GRAS | 1° flavor | David Michael; Citrus |
| WONF) | | | profile matches low pH |
| Lime (Natural and | GRAS | 2° flavor | FONA; Single flavor |
| WONF) | | | supplier |
| Lemon (Natural and | GRAS | 2° flavor | FONA; Single flavor |
| artificial) | | | supplier |
| Taste Modifier | GRAS | Bitterness masking | FONA; Useful at low |
| | | | volume |
| FD&C Yellow No. 6 | USP | Color | Sensient; Match flavor |
| | | | profile |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 4).

TABLE 4
Exemplary contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Production of Dry Blended Preparations

To produce the dry blended preparations of the instant disclosure, the following general steps may be used: individual pharmaceutical grade amino acid entities (and, optionally, one or more excipients and/or oral administration components), may be combined into a combination and subjected to one or more blending conditions (e.g., blending and mixing). In some embodiments, the blending conditions are continued until the combination meets one or more reference standards. In some embodiments, the resulting PGDBP is divided into a plurality of portions. In some embodiments, at least a percentage of the portions of the plurality of portions also meet one or more reference standards, e.g., the reference standards that the PGDBP met. In some embodiments, at least a percentage of the portions of the plurality of portions meet one or more reference standards.

In some embodiments, the dry blended preparation, e.g., PGDBP, is also a large-scale preparation. Large-scale, as used herein, describes a preparation that is larger (e.g., by weight, mass, or volume) than a reference value. In some embodiments, the reference value is the size of a typical experimental (e.g., non-manufacturing) preparation. In some embodiments, the reference value is 10, 11, 12, 13, 14, or 15 kg. In some embodiments, large-scale preparations comprise at least 25, 30, 40, 50, 60, 70, 80, 90, 100, 120, 140, 160, 180, 200, 300, 400, 500, 600, 700, 800, 900, 1000, 2000, 3000, 4000, 5000, 6000, 7000, 8000, 9000, or 10000 kg. In some embodiments, large-scale preparations comprise no more than 10000, 5000, 1000, 900, 800, 700, 600, 500, 400, or 300 kg. In some embodiments, a large-scale preparation comprises 100-500 kg, 100-400 kg, 100-300, 100-200 kg, 200-300 kg, 200-400 kg, 200-500 kg, 300-400 kg, 300-500 kg, 400-500, or 500-1000 kg.

### Blending Techniques

The methods disclosed herein comprise blending steps which blend and mix combinations of pharmaceutical grade amino acid entities to create PGDBPs that meet a reference standard. Blending conditions used by the methods described herein may utilize any known blending mechanism or combination of blending mechanisms. Blending mechanisms include convection, diffusion, and shear. Convective blending utilizes gross motion of particles, e.g., by gentle rotation within a blender/mixer. Diffusion is the slow, passive blending of particles. Shear blending pushes part of a combination of particles in one direction and another part of the combination of particles in another direction along the same parallel plane. Blending conditions used by the methods described herein may further comprise the use of granulators or other equipment to modify the size and/or shape of particles of combination components (e.g., pharmaceutical grade amino acid entities).

In some embodiments, the blending or blending condition employed by a method disclosed herein comprises convective blending. In some embodiments, the blending or blending condition employed by a method disclosed herein comprises diffusion blending. In some embodiments, the blending or blending condition employed by a method disclosed herein comprises shear blending. In some embodiments, the blending or blending condition employed by a method disclosed herein comprises convective and diffusion blending. In some embodiments, the blending or blending condition employed by a method disclosed herein comprises convective and shear blending. In some embodiments, the blending or blending condition employed by a method disclosed herein comprises diffusion and shear blending. In some embodiments, the blending or blending condition employed by a method disclosed herein comprises convective, diffusion, and shear blending.

Blending conditions used by the methods described herein may utilize any known blending or mixing equipment; blending or mixing equipment may operate based on one or more blending mechanisms. There are four main types of blending or mixing equipment: convective, hoppers (i.e., gravimetric), tumblers, and fluidization. In some embodiments, a blending condition or blending step of a method described herein may utilize one or more (e.g., 1, 2, 3, or 4) types of blending or mixing equipment. In some embodiments, dry blended preparations (e.g., PGDBPs) are prepared in batches. In some embodiments, dry blended preparations (e.g., PGDBPs) are prepared in a continuous fashion, e.g., harvesting blended/mixed preparation without interrupting blending or mixing.

The blending or mixing steps of methods disclosed herein are of duration sufficient to produce a dry blended preparation, e.g., PGDBP, which meets a reference standard. In some embodiments, the duration of the blending condition is at least 10, 15, 20, 25, 30, 35, 40, 45, 50, 55, 60, 75, 90, 105, or 120 minutes. In some embodiments, the duration of the blending condition is no more than 180, 165, 150, 135, 120, 105, 90, 75, 60, 55, 50, 45, 40, 35, 30, 25, or 20 minutes. In some embodiments, the duration of the blending condition is 20-90, 20-60, 20-50, 20-40, 20-30, 30-90, 30-60, 30-50, 30-40, 40-90, 40-60, 40-50, 50-90, 50-60, or 60-90 minutes. In some embodiments, the duration of the blending condition is 20-40 minutes, e.g., 20 minutes, 30 minutes, or 40 minutes. In some embodiments, the duration of the blending condition is sufficient that blending and mixing does not introduce heterogeneity into the combination or dry blended preparation, e.g., by over-mixing. In some embodiments, the duration of the blending condition is determined by evaluation of whether a reference standard has been met. For example, the blending condition may continue until an evaluation shows that the reference standard has been met. In some embodiments wherein the reference standard is composition uniformity, e.g., blend uniformity, evaluating whether a reference standard has been met comprises using near infrared spectroscopy (NIR). In an embodiment, the blending condition is maintained until the NIR spectrum observed shows that a standard for composition uniformity, e.g., blend uniformity, has been met.

In some embodiments, the methods disclosed herein comprise blending steps which blend and mix combinations of pharmaceutical grade amino acid entities to create PGDBPs, wherein the blending steps occur at room temperature, e.g., between 15 and 35° C., e.g., between 20 and 30° C., e.g., at about 25° C. In some embodiments, the blending steps occur at a temperature lower than 20, 21, 22, 23, 24, 25, 26, 27, 28, 29, 30, 31, 32, 33, 34, 35, 36, 37, 38, 39, or 40° C. (and optionally, at a temperature of at least 5, 6, 7, 8, 9, 10, 11, 12, 13, 14, 15, 16, 17, 18, 19, 20, 21, 22, 23, 24, or 25° C.). In some embodiments, the blending steps occur at a temperature of about 20, 21, 22, 23, 24, 25, 26, 27, 28, 29, or 30° C.

In some embodiments, the methods disclosed herein comprise blending steps which blend and mix combinations of pharmaceutical grade amino acid entities to create PGDBPs, wherein the blending steps comprise use of a blender or mixer rotation speed (e.g., a blender or mixer rotor rotational speed) of less than 10,000, 9,000, 8,000, 7,000, 6,000, 5,000, 4,000, 3,000, 2,000, 1,000, 500, 250, 100, 90, 80, 70, 60, 50, 40, 30, 20, or 10 rotations per minute (rpm) (and optionally, at least 5, 10, 15, 20, 25, 30, 35, 40, 45, 50, 60, 70, 80, 90, or 100 rpm). In some embodiments, the blending steps comprise use of a blender or mixer rotation speed (e.g., a blender or mixer rotor rotational speed) of about 20, 30, 40, 50, 60, 70, 80, 90, or 100 rpm. In some embodiments, the blending steps comprise use of a blender or mixer rotation speed (e.g., a blender or mixer rotor rotational speed) of between 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 rpm.

In some embodiments, the method further comprises roller compaction and/or wet granulation. In some embodiments, the method further comprises automated filling, e.g., which incorporates direct blending, roller compaction, or wet granulation.

Segregation of different species of particles in a combination (e.g., dry blended preparation, e.g., PGDBP) during blending or mixing, division of portions, or downstream processing is a barrier to meeting and maintaining reference standards, e.g., a standard of composition uniformity. Any mixture of two or more types of particles can be vulnerable to segregation. Segregation can occur by one or more of several mechanisms, including sifting, fluidization, and dusting (e.g., see Purutyan, H, and Carson, J. W. Predicting, diagnosing, and solving mixture segregation problems. Jenike & Johnson, CSC Publishing, Powder and Bulk Engineering, 2013).

### Sampling and Measurement

The methods described herein for manufacturing a dry blended preparation, e.g., a PGDBP, that meets a reference standard may further comprise evaluating whether the reference standard has been met. In some embodiments, the methods described herein comprise acquiring a value, e.g., for the amount of a pharmaceutical grade amino acid entity, from one or more sampling points in a dry blended preparation, e.g., PGDBP. A sampling point is a location, e.g., defined spatially and temporally, within a dry blended preparation, e.g., PGDBP. In some embodiments, to acquire a value, a sampling point may be accessed. Accessing a sampling point may comprise using a diagnostic technique on the dry blended preparation of the sampling point. In some embodiments, accessing, e.g., using a diagnostic technique, comprises stopping or pausing the blending or mixing or blending condition to access the sampling point. In some embodiments, accessing, e.g., using a diagnostic technique, does not comprise stopping or pausing the blending or mixing or blending condition to access the sampling point. Sampling points may be designated and/or accessed by methods known in the art.

In some embodiments, samples acquired from a sampling point of a combination or dry blended preparation (e.g., PGDBP) or portions of a dry blended preparation (e.g., PGDBP) may be analyzed using near-infrared (NIR) spectroscopy to acquire a value (e.g., for composition uniformity, e.g., blend uniformity). NIR spectroscopy analyzes the absorption spectra of compounds in the NIR wavelength region (780-2500 nm). Absorption peaks of compounds, e.g., pharmaceutical grade amino acid entities, are produced by molecular vibrations classified into two types: overtones and combinations. Compounds comprising CH, OH, or NH bonds can be analyzed using NIR. Methods of interpreting NIR spectra are known in the art. In some embodiments, NIR spectroscopy is used to determine whether the amounts of amino acid entities at a plurality of sampling points are similar, e.g., whether a standard for homogeneity (e.g., composition uniformity, e.g., blend uniformity) has been met. In some embodiments, the methods further comprise, responsive to the determination, selecting and/or executing a step, e.g., selecting and using a blending or mixing technique or blending condition or ending blending, mixing, or a blending condition.

In some embodiments, samples acquired from a sampling point of a combination or dry blended preparation (e.g., PGDBP) or portions of a dry blended preparation (e.g., PGDBP) may be analyzed using high performance liquid chromatography (HPLC, also referred to as high-pressure liquid chromatography) to acquire a value (e.g., for the amount of a pharmaceutical grade amino acid entity).

In some embodiments, samples acquired from a sampling point of a combination or dry blended preparation (e.g., PGDBP) or portions of a dry blended preparation (e.g., PGDBP) may be analyzed using liquid chromatography mass spectrometry (LC-MS). In some embodiments, LC-MS is used to determine the identity and/or amounts of pharmaceutical grade amino acid entities present at a sampling point or in a portion. In some embodiments, LC-MS is used to determine whether a dry blended preparation meets a standard for composition uniformity, e.g., portion or blend uniformity. In some embodiments, the methods further comprise, responsive to the amount(s) of pharmaceutical grade amino acid entities present, selecting and/or executing a step, e.g., selecting and using a blending or mixing technique or blending condition or ending blending, mixing, or a blending condition.

### Reference Standards

The methods described herein produce dry blended preparations, e.g., PGDBPs, which meet one or more reference standards. A reference standard, as used herein, means: a standard used or set by:

(1) a manufacturer of a combination (e.g., dry blended preparation, e.g., PGDBP), e.g., a manufacturer having approval from a governmental agency to market the PGDBP, or

(2) the pharmaceutical industry or agencies or entities (e.g., government or trade agencies or entities) regulating the pharmaceutical industry,

to ensure one or more product quality parameters are within acceptable ranges for a medicine, pharmaceutical composition, treatment, or other therapeutic. A product quality parameter can be any parameter regulated by the manufacturer, pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA), Health Canada, and Medicines and Healthcare Products Regulatory Agency (MHRA). A product quality parameter can also be a parameter specified by a national or regional pharmacopeia or formulary, including the U.S. Pharmacopeia (USP), British Pharmacopeia (BP), National Formulary (NF), European Pharmacopeia (EP), Japanese Pharmacopeia (JP), or the International Council for Harmonisation of Technical Requirements for Pharmaceuticals for Human Use (ICH).

The one or more reference standards may be a standard used or promulgated by the pharmaceutical industry or by agencies or entities, e.g., government or trade agencies or entities, regulating the pharmaceutical industry to ensure one or more product quality parameters are within acceptable ranges for a medicine, pharmaceutical composition, treatment, or other therapeutic. The one or more reference standards may be a standard used or set by a manufacturer of a combination (e.g., dry blended preparation, e.g., PGDBP), e.g., a manufacturer having approval from a governmental agency to market the PGDBP, to ensure one or more product quality parameters are within acceptable ranges for a supplement, nutriceutical, medicine, pharmaceutical composition, treatment, or other therapeutic. A product quality parameter can be any parameter regulated by the manufacturer, or by the pharmaceutical industry or by agencies or entities, e.g., government or trade agencies or entities, including but not limited to composition; composition uniformity; dosage; dosage uniformity; presence, absence, and/or level of contaminants or impurities; level of sterility (e.g., the presence, absence and/or level of microbes), color, or particle morphology (e.g., size or shape).

### Composition Uniformity

In some embodiments, the reference standard is composition uniformity. Composition uniformity, in general, is a standard of homogeneity. Composition uniformity can be classified into two different but related types of uniformity: blend uniformity and portion uniformity (portion uniformity is used interchangeably with content uniformity and dosage uniformity herein). Composition uniformity may comprise one or both types depending on the usage and context. Composition uniformity may comprise a standard of the homogeneity of a combination (e.g., dry blended preparation, e.g., PGDBP) with regards to one or a plurality of components. In some embodiments, a combination that meets a standard for composition uniformity does so with regards to one, two, three, four, or more (e.g., all) components (e.g., pharmaceutical grade amino acid entities).

Blend Uniformity

Blend uniformity refers to the level of homogeneity of the distribution of components in a combination, e.g., dry blended preparation, e.g., PGDBP. In some embodiments, a standard for composition uniformity, e.g., blend uniformity, is met when the amount of a component (e.g., a pharmaceutical grade amino acid entity) at a first sampling point in the combination (e.g., dry blended preparation, e.g., PGDBP) differs by no more than a predetermined amount from a reference value. Amounts may be absolute, e.g., grams, or relative, e.g., weight/weight (e.g., X g of the component in Y g of sampling point). Amounts may be arbitrary values, as in the case of comparing absorbance values to absorbance values or in statistical comparisons of curves, e.g., of spectra. In some embodiments, acquiring a value for blend uniformity comprises assessing a standard for composition uniformity, e.g., blend uniformity, by acquiring a value for the amount of a component at a first sampling point in the combination and comparing it to reference value.

In some embodiments, NIR is used to determine whether the amount of a component (e.g., a pharmaceutical grade amino acid entity) at a first sampling point in the combination (e.g., dry blended preparation, e.g., PGDBP) differs by no more than a predetermined amount from a second or further sampling. Using NIR, the near infrared spectrum for a sampling point can be acquired and compared to the near infrared spectrum for a second or further sampling point (e.g., a third, fourth, fifth, sixth, seventh, eighth, ninth, and/or tenth sampling point) or to the near infrared spectrum for a sample known to meet a reference standard, e.g., a standard for composition uniformity, e.g., blend uniformity. If the comparison shows that the spectra are similar enough to one another, a standard for blend uniformity is met. Similarity of NIR spectra can be evaluated by comparing the conformity index of sampling points. The conformity index is a value generated by the NIR spectra obtained, and the examples of conformity indices described are not an exhaustive list of all possible conformity indices. The conformity index may be the absorbance at a particular wavelength or wavelengths in the near infrared range. The conformity index may be the standard deviation of the average absorbance at a particular wavelength or wavelengths in the near infrared range at a plurality of sampling points. The key characteristic of the conformity index, whichever value is selected, is that the conformity indices of the sampling points accessed converge (in the case of absorbance at particular wavelength) or reduce (in the case of standard deviation) as blending/mixing time increases. For example, the conformity index may be selected to be a wavelength of X nm in the near infrared range. The absorbance at X nm will be measured at a plurality of sampling points at time points during blending. As blending continues, the absorbance at X nm at each sampling point will grow more similar to one another.

In some embodiments, the reference value is the amount of the component at a second or further sampling point (e.g., a third, fourth, fifth, sixth, seventh, eighth, ninth, and/or tenth sampling point) sampling point in the combination (e.g., dry blended preparation, e.g., PGDBP). The second sampling or further sampling point (e.g., a third, fourth, fifth, sixth, seventh, eighth, ninth, and/or tenth sampling point) point may be a different spatial location in the combination, for example, samples can be collected from a set of predetermined, spread out spatial locations, e.g., a stratified sampling plan with predetermined sites to be sampled, e.g., to obtain samples that represent a variety of locations in the blender or mixer.

In some embodiments, the second sampling point is 1, 2, 3, 4, 5, 6, 7, 8, 9, 10, or more minutes after the first sampling point. In some embodiments, multiple sampling points separated in time are taken throughout the process of manufacturing the dry blended preparation (e.g., PGDBP). In some embodiments, the sampling points separated in time are at intervals throughout the process of manufacturing the dry blended preparation (e.g., PGDBP), e.g., every 1, 2, 3, 4, 5, 6, 7, 8, 9, or 10 minutes. In some embodiments, the multiple sampling points are compared to one another (e.g., the most recent sampling points are compared to each other).

In some embodiments, a standard for composition uniformity, e.g., blend uniformity, is met when the amount of the component at a first sampling point differs from the reference value, e.g., the amount of the component at a second or further sampling point (e.g., a third, fourth, fifth, sixth, seventh, eighth, ninth, and/or tenth sampling point) by less than or equal to 1, 2, 3, 4, 5, 6, 7, 8, 9, or 10%, e.g., 10%. In some embodiments, a standard for composition uniformity is met when the amount of a component at a first sampling differs by no more than 10% from the amount of the component at a second or further sampling point (e.g., a third, fourth, fifth, sixth, seventh, eighth, ninth, and/or tenth sampling point). In some embodiments, a standard for composition uniformity is met when the amount of a component at a first sampling differs by no more than 10% from the amount of the component present in the combination (e.g., dry blended preparation, e.g., PGDBP). In some embodiments, a standard for composition uniformity is met when the amount of a component at the most recent sampling point differs by no more than 10% from the amount of the component present at the next most recent sampling point. Values for the amount of a component present at a sampling point can comprise NIR spectra. Comparisons of values for the amount of a component present at a first, second, or further sampling point can comprise comparison of NIR spectra, e.g., overlaying NIR spectra or comparing conformity indices of the first, second, or further sampling points. Blend uniformity can be met when NIR spectra, e.g., conformity indices, reach a threshold of similarity or overlap.

Portion Uniformity

Portion uniformity refers to the homogeneity of portions of the dry blended preparation, e.g., PGDBP, with respect to amounts of components (e.g., pharmaceutical grade amino acid entities). In some embodiments, the methods described herein comprise division of a dry blended preparation (e.g., PGDBP) into a plurality of portions. In some embodiments, a standard for composition uniformity, e.g., portion uniformity, is met when the amount of a component (e.g., a pharmaceutical grade amino acid entity) in a first portion differs by no more than a predetermined amount from a reference value. Amounts may be absolute, e.g., grams, or relative, e.g., weight/weight (e.g., X g of the component in Y g of sampling point). In some embodiments, the amount of a a component (e.g., a pharmaceutical grade amino acid entity) in a first, second, or further portion (e.g., a third, fourth, fifth, sixth, seventh, eighth, ninth, or tenth portion) is determined using HPLC.

In some embodiments, the reference value is the amount of the component in a second portion. In some embodiments, the reference value is the amount(s) of the component in a plurality of portions, e.g., a plurality of test portions (e.g., the first portion is compared to a plurality of test portions). In an embodiment, the reference value is the average or median amount of the component in the plurality of test portions.

In some embodiments, a standard for composition uniformity, e.g., portion uniformity, is met when the amounts of a component (e.g., a pharmaceutical grade amino acid entity) in a plurality of test portions differ by no more than a predetermined amount from a reference value. Amounts may be absolute, e.g., grams, or relative, e.g., weight/weight (e.g., X g of the component in Y g of sampling point). In some embodiments, the reference value is the average or median amount of the component in the plurality of test portions.

In some embodiments, the reference value is the amount of the component in the combination (e.g., dry blended preparation, e.g., PGDBP). For example, the reference value can be overall weight/weight of the component present in the total combination. In some embodiments, evaluating whether a standard for composition uniformity is met comprises comparing a relative amount of a component at a first sampling point (e.g., X g of the component in Y g of sampling point) to the relative amount of the component in the combination (e.g., W g of the component in Z g of combination total); in other words, evaluating the standard for composition uniformity may comprise comparing X/Y to W/Z.

In an embodiment, at least X % of the portions of the plurality of portions of the dry blended preparation (e.g., PGDBP) are test portions, wherein X is 1, 2, 3, 4, 5, 6, 7, 8, 9, 10, 15, 20, 25, 30, 35, 40, 45, or 50. In some embodiments, no more than X % of the portions of the plurality of portions of the dry blended preparation (e.g., PGDBP) are test portions, wherein X is 50, 45, 40, 35, 30, 25, 20, 15, 10, 9, 8, 7, 6, 5, 4, 3, 2, or 1. In an embodiment, test portions are portions compared to a reference value, e.g., one another or the amount of a component present in the dry blended preparation (e.g., PGDBP), to determine whether a reference standard (e.g., for composition uniformity, e.g., portion uniformity) has been met. In some embodiments, a standard for composition uniformity, e.g., portion uniformity, is met when the amount of a component present in at least X % of test portions differs from a reference value by no more than 1, 2, 3, 4, 5, 6, 7, 8, 9, or 10%, wherein X is 50, 60, 70, 80, 85, 90, 95, 99, or 100%, and wherein the reference value is selected from the average amount of the component present in the test portions, the median amount of the component present in the test portions, or the amount of the component present in the dry blended preparation (e.g., PGDBP).

In some embodiments, portions of the dry blended preparation (e.g., PGDBP) may be stick packs or other unit dosage forms.

### Level of Contamination

In some embodiments, the reference standard is level of contamination. When combining raw materials, e.g., pharmaceutical grade amino acid entities and/or excipients, into a combination, e.g., dry blended preparation, e.g., PGDBP, contaminants may be present in the combination. A combination, e.g., dry blended preparation, e.g., PGDBP, meets a standard for level of contamination when the combination does not substantially comprise (e.g., comprises less than 10, 9, 8, 7, 6, 5, 4, 3, 2, 1, 0.15, 0.1, 0.05, 0.01, or 0.001% (w/w) of) a contaminant. In some embodiments, a combination, e.g., dry blended preparation, e.g., PGDBP, comprises less than 0.15% (w/w) of a contaminant. In some embodiments, a combination, e.g., dry blended preparation, e.g., PGDBP, comprises a lower level of a contaminant than the level permissible in food (e.g., as defined by appropriate regulatory organizations known in the art). In some embodiments, a combination, e.g., dry blended preparation, e.g., PGDBP, described in a method herein does not comprise a contaminant. Contaminants include any substance that is not deliberately present in the combination, e.g., dry blended preparation, e.g., PGDBP, (for example, pharmaceutical grade amino acid entities and excipients, e.g., oral administration components, are deliberately present) or any substance that has an unintended negative effect on a product quality parameter of the PGDBP or plurality of portions of PGDBP (e.g., side effects in a subject, decreased potency, decreased stability/shelf life, discoloration, odor, bad taste, bad texture/mouthfeel, or increased segregation of components of the PGDBP). In some embodiments, contaminants include microbes, endotoxins, metals (e.g., heavy metals), residual solvents, raw material impurities, extractables, and/or leachables. In some embodiments, a combination, e.g., dry blended preparation, e.g., PGDBP, comprises a level of contaminant (e.g., does not substantially comprise a contaminant) that is compliant with a reference standard, e.g., a standard promulgated by an agency known to those of skill in the art or described herein. In some embodiments, a combination, e.g., dry blended preparation, e.g., PGDBP, comprises a level of contaminant (e.g., does not substantially comprise a contaminant) that is compliant with a standard of the ICH, e.g., the ICH Q3A Impurities in New Drug Substances standard.

In some embodiments, the methods described herein further comprise acquiring a value for the level of a contaminant at a sampling point in one or both of the combination or PGDBP. In some embodiments, the methods described herein further comprise acquiring a value for the level of a contaminant at each of a plurality of points in one or both of the combination or PGDBP, or in a test portion (e.g., of the combination or PGDBP). In some embodiments, the methods described herein further comprise acquiring a value for the level of a contaminant in a portion, e.g., a test portion, of the plurality of portions. In some embodiments, responsive to the value for the level of the contaminant, e.g., and determining that a standard for the level of contamination is met, the methods described herein further comprise selecting and executing a downstream processing step, e.g., dividing the PGDBP into portions (e.g., portioning) and fill-finish (e.g., formulation (e.g., with excipients), packaging, and labeling) and distribution. In some embodiments, responsive to the value for the level of the contaminant, e.g., and determining that a standard for the level of contamination is not met, the methods described herein further comprise selecting and executing a different downstream processing step, e.g., purification and/or removal of the contaminant or disposal of the portion, plurality of portions, or PGDBP.

### Dietary Compositions

The composition (e.g., the Active Moiety) including amino acid entities can be formulated and used as a dietary composition, e.g., chosen from a medical food, a functional food, or a supplement. In such an embodiment, the raw materials and final product should meet the standards of a food product. The composition of any of the aspects and embodiments disclosed herein can be for use as a dietary composition, e.g., chosen from a medical food, a functional food, or a supplement. In some embodiments, the dietary composition is for use in a method, comprising administering the composition to a subject. The composition can be for use in a dietary composition for the purpose of improving liver function or a liver disease or disorder.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of a liver disease or disorder (e.g., in a subject with a liver disease or disorder).

The present disclosure features a method of improving a liver disease or disorder comprising administering to a subject an effective amount of a dietary composition described herein.

The present disclosure features a method of providing nutritional support or supplementation to a subject with a liver disease or disorder (e.g., a subject with a liver disease or disorder), comprising administering to the subject an effective amount of a composition described herein.

The present disclosure features a method of providing nutritional support or supplementation that aids in the management of a liver disease or disorder (e.g., a liver disease or disorder), comprising administering to a subject in need thereof an effective amount of a composition described herein.

In some embodiments, the method is a non-therapeutic method, e.g., one, two, or three of increasing fat metabolism for weight loss, of maintaining health, or for cosmetic purposes.

In some embodiments, the subject has or has been diagnosed with a liver disease or disorder. In other embodiments, the subject does not have a liver disease or disorder.

The compositions can be used in methods of dietary management of a subject (e.g., a subject without a liver disease or disorder).

In some embodiments, the subject has a fatty liver disease or disorder.

In some embodiments, the fatty liver disease or disorder is chosen from: non-alcoholic fatty liver disease (NAFLD) or alcoholic fatty liver disease (AFLD). In certain embodiments, the NAFLD is chosen from NASH or non-alcoholic fatty liver NAFL. In certain embodiments, the subject has pediatric NAFLD. In certain embodiments, the AFLD is ASH.

In some embodiments, the subject has one or both of fibrosis or steatosis.

In certain embodiments, the subject (e.g., a subject with NASH) has cirrhosis. In some embodiments, the subject has hepatocarcinoma. In certain embodiments, the subject has one or both of an increased risk of liver failure or an increased risk of death.

In some embodiments, the subject has one, two, three, or more (e.g., all) of diabetes (e.g., type 2 diabetes), metabolic syndrome, a relatively high BMI, or obesity.

In some embodiments, the composition promotes weight loss in the subject.

In some embodiments, the subject has one, two, or more (e.g., all) of gut leakiness, gut dysbiosis, or gut microbiome disturbance.

In some embodiments, the subject has diabetic peripheral neuropathy.

### Biomarkers

Any of the methods disclosed herein can include evaluating or monitoring the effectiveness of administering a composition of the invention as described herein to a subject with a liver disease or disorder (e.g., NAFLD (e.g., NASH or NAFL) or AFLD (e.g., ASH)). The method includes acquiring a value of effectiveness to the composition, such that the value is indicative of the effectiveness of the therapy.

In some embodiments, the value of effectiveness of the composition in treating a subject with a liver disease (e.g., NAFLD (e.g., NASH or NAFL) or AFLD (e.g., ASH)) comprises a measure of one, two, three, four, five, six, seven, eight, nine, 10, 11, 12, 13, 14, 15, 16, 17, 18, 19, 20, 21, 22, or more (e.g., all) of: a) alanine aminotransferase (ALT); b) aspartate aminotransferase (AST); c) adiponectin; d) N-terminal fragment of type III collagen (proC3); e) caspase-cleaved keratin 18 fragments (M30 and M65); f) IL-1β; g) C-reactive protein; h) PIIINP; i) a tissue inhibitor of metalloproteinase (TIMP); e.g., TIMP1 or TIMP2; j) MCP-1; k) FGF-21; l) Col1a1; m) Acta2; n) a matrix metalloproteinase (MMP), e.g., MMP-13, MMP-2, MMP-9, MT1-MMP, MMP-3, or MMP-10; o) ACOX1; p) IL-10; q) NF-kB; r) TNF-α; s) hydroxyproline; t) IL-2; u) MIP-1; v) α-SMA; or w) TGF-β.

In some embodiments, the subject exhibits increased levels of one, two, three, four, five, six, seven, eight, nine, 10, 11, 12, 13, 14, 15, 16, 17, 18, 19, 20, or more (e.g., all) of: ALT; AST; proC3; caspase-cleaved keratin 18 fragments (M30 and M65); IL-1β; C-reactive protein; PIIINP; a TIMP (e.g., TIMP1 or TIMP2); MCP-1; FGF-21; Col1a1; Acta2; a MMP (e.g., MMP-13, MMP-2, MMP-9, MT1-MMP, MMP-3, or MMP-10); ACOX1; NF-kB; TNF-α; hydroxyproline; IL-2; MIP-1; α-SMA; or TGF-β, e.g., relative to a healthy subject without a liver disease or disorder. In some embodiments, the subject exhibits one or both of decreased levels of IL-10 or adiponectin, e.g., relative to a healthy subject without a liver disease or disorder.

In some embodiments, administration of the composition (e.g., at a dosage regimen described herein) to the subject reduces the level or activity of one, two, three, four, five, six, seven, eight, nine, 10, 11, 12, 13, 14, 15, 16, 17, 18, 19, 20, or more (e.g., all) of: ALT; AST; proC3; caspase-cleaved keratin 18 fragments (M30 and M65); IL-1β; C-reactive protein; PIIINP; a TIMP (e.g., TIMP1 or TIMP2); MCP-1; FGF-21; Col1a1; Acta2; a MMP (e.g., MMP-13, MMP-2, MMP-9, MT1-MMP, MMP-3, or MMP-10); ACOX1; NF-kB; TNF-α; hydroxyproline; IL-2; MIP-1; α-SMA; or TGF-β. In some embodiments, administration of the composition to the subject increases the level or activity of one or both of IL-10 or adiponectin.

### EXAMPLE

The Example below is set forth to aid in the understanding of the inventions, but is not intended to, and should not be construed to, limit its scope in any way.

### Example 1. Hepatocyte Model for Steatosis and Inflammation

Hepatocyte lipotoxicity appears to be a central driver of hepatic cellular injury via oxidative stress and endoplasmic reticulum (ER) stress. The ability of amino acids to influence steatosis (lipid accumulation) and inflammation in hepatocytes was assessed using human primary hepatocytes (Sekisui Xenotech).

### Cell Seeding and Maintenance

Primary hepatocytes lot nos. from one healthy human donor were seeded on day 0 at density of 6E+04 cells in 96 well optical microplates (Thermofisher) in hepatocyte plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO₂. After 6 hours, cells were washed twice and incubated overnight at 37° C., 5% CO₂ in hepatocytes plating media. On day 1, cells were washed twice and incubated for 24 h in Hepatocytes defined medium (Corning) supplemented with 2 mM Glutamax (Gibco), and 1× Penicillin/Streptomycin in the same conditions described above.

### Amino Acids Pre-Treatment

On day 2, cells were washed twice with DPBS 1× (Gibco) and maintained in amino acid-free WEM (US Biologicals) containing a defined custom amino acid concentration based on the mean physiological concentrations in blood. The values are published in the Human Metabolome Database (Wishart D S, Tzur D, Knox C, et al., HMDB: the Human Metabolome Database. Nucleic Acids Res. 2007 January; 35(Database issue):D521-6. 17202168; which is hereby incorporated by reference in its entirety). This custom media is supplemented with 11 mM Glucose, 0.272 mM Sodium Pyruvate, and a dose curve of defined amino acid compositions (i.e., vehicle, LIRNAC+/−L-carnitine) at various ranges of concentrations. Cells were maintained in this defined media for 24 hours at 37° C., 5% CO₂.

Co-Treatment with Free Fatty Acids and Different Amino Acids Combination

After pre-treatment, cells were exposed to free fatty acids (FFA) at 250 uM with a ratio of 2:1 (Oleate:Palmitate) supplemented with TNF-α (Thermofisher) at 1 ng/ml or vehicle. Cells were incubated with the FFAs mixture and the different amino acids combinations for 24 hours at 37° C., 5% CO₂. After 24 hours incubation, media was removed for cytokine analysis and replaced by fresh media containing the same stimulus conditions and amino acid concentrations. Cells were incubated for an additional 48 hours for a total of 72 hours of FFA and TNF-α stimulation.

Cytokine Analysis after 24 h by ELISA

Human CCL2 (MCP-1) was measured by ELISA (Human CCK2/MCP-1 DuoSet ELISA, R&D Systems) at 1/10 dilution in 1× Reagent Diluent (Reagent Ancillary Kit 2, R&D Systems). Statistical analysis was done using one-way ANOVA.

Intracellular Lipid Accumulation Analysis after 72 h by Fluorescence Microscopy

After 72 hours, cells were washed twice with PBS 1× (Gibco), fixed with 4% Paraformaldehyde, and washed twice with PBS 1× (100 ul). After fixation, lipids were stained with HCS LipidTOX Red Neutral (Thermofisher Scientific) diluted 1000× and nuclei were stained with Hoechst 3342 (Life Technologies) diluted to 4 ug/ml. The LipidTOX™ neutral lipid stain has an extremely high affinity for neutral lipid droplets that was detected by fluorescence microscopy using a high content imager (Molecular Devices). Data was normalized to the specific per well cell density determined by nuclei count. Images were analyzed and total lipid area was calculated using MetaXpress 6 software. Statistical analysis was done using one-way ANOVA.

### Results

### Lipid Accumulation and Steatosis Phenotypes

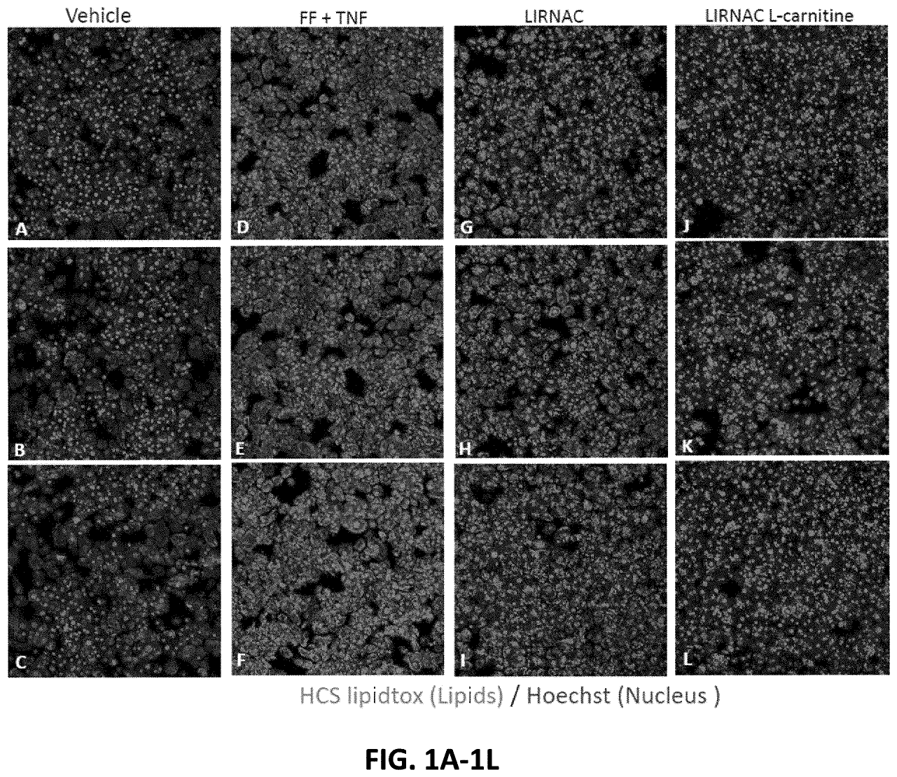

Table 5 shows the level of total lipid area normalized to nuclear count and baseline subtracted in primary human hepatocytes cells from one healthy donor 1. Primary human hepatocytes from healthy donors were found to have low levels of lipid accumulation (FIG. 1A-1C, Table 5 row 11). Treatment of the cells with free fatty acids (FF)+TNFα induced lipid accumulation (FIG. 1D-1F, Table 5 row 5, 10) with a macro-steatosis phenotype. Treatment with LIRNAC changes hepatocytes lipid phenotype from macro-steatosis to micro-steatosis with no effect on total lipid area (FIG. 1G-1I, Table 5 row 1-4). In addition to changing lipid phenotype to micro-steatosis, treatment with the combination LIRNAC+L-carnitine reduces significantly total lipid area (FIG. 1J-1L, Table 5 row 6-9) in a dose dependent manner.

TABLE 5
Changes in total lipid area for donor 1 upon administration of amino acid compositions

| | | | | | Total lipid area relative to Control - Donor 1 | | | | |
|---|---|---|---|---|---|---|---|---|---|
| | | Amino Acid | AA Conc. | L-carnitine | | Std. | Number | | |
| Row | Treatment | Supplement | (X) | (uM) | Mean | Deviation | of values | P-value* | Significance |
| 1 | FFA + TNF | LIRNAC | 40 | 0 | 0.96 | 0.06 | 3 | 08721 | ns |
| 2 | FFA + TNF | LIRNAC | 30 | 0 | 1.03 | 0.09 | 3 | 0.9314 | ns |
| 3 | FFA + TNF | LIRNAC | 20 | 0 | 1.08 | 0.09 | 3 | 0.3751 | ns |
| 4 | FFA + TNF | LIRNAC | 10 | 0 | 0.98 | 0.01 | 3 | 0.9915 | ns |
| 5 | FFA + TNF | HMDB* | 1 | 0 | 1 | 0.02 | 3 | | |
| 6 | FFA + TNF | LIRNAC + L-carnitine | 40 | 500 | 0.55 | 0.03 | 3 | 0.0001 | **** |
| 7 | FFA + TNF | LIRNAC + L-carnitine | 30 | 375 | 0.75 | 0.10 | 3 | 0.0002 | *** |
| 8 | FFA + TNF | LIRNAC + L-carnitine | 20 | 250 | 0.82 | 0.03 | 3 | 0.0029 | ** |
| 9 | FFA + TNF | LIRNAC + L-carnitine | 10 | 10 | 0.86 | 0.03 | 3 | 0.0145 | * |
| 10 | FFA + TNF | HMDB* | 1 | 0 | 1 | 0.02 | 3 | | |
| 11 | No FFA No TNF | HMDB* | 1 | 0 | 0.45 | 0.02 | 3 | 0.0001 | **** |

*HMDB: amino acid concentration based on the mean physiological concentrations in blood. The values are published in the Human Metabolome Database (HMDB).

### MCP1/CCL2 Secretion

Table 6 shows the baseline subtracted secretion of MCP1/CCL2 in primary human hepatocytes cells from one healthy donor 1. LIRNAC and LIRNAC+L-carnitine significantly decrease MCP1/CCL2 secretion in a dose dependent manner.

TABLE 6
Changes in MCP1 expression for donor upon administration of amino acid compositions

| | | | | MCP1 expression relative to Control - Donor 1 | | | | |
|---|---|---|---|---|---|---|---|---|
| | Amino Acid | AA | L-carnitine | | Std. | Number | | |
| Treatment | Supplement | Conc. (X) | (uM) | Mean | Deviation | of values | P-value* | Significance |
| FFA + TNF | LIRNAC | 40 | 0 | 0.06 | 0.03 | 3 | 0.0001 | **** |
| FFA + TNF | LIRNAC | 30 | 0 | 0.12 | 0.004 | 3 | 0.0001 | **** |
| FFA + TNF | LIRNAC | 20 | 0 | 0.27 | 0.05 | 3 | 0.0001 | **** |
| FFA + TNF | LIRNAC | 10 | 0 | 0.67 | 0.21 | 3 | 0.0119 | * |
| FFA + TNF | HMDB* | 1 | 0 | 1.00 | 0.08 | 3 | | |
| FFA + TNF | LIRNAC + L-carnitine | 40 | 500 | 0.03 | 0.006 | 3 | 0.0001 | **** |
| FFA + TNF | LIRNAC + L-carnitine | 30 | 375 | 0.11 | 0.06 | 3 | 0.0001 | **** |
| FFA + TNF | LIRNAC + L-carnitine | 20 | 250 | 0.25 | 0.03 | 3 | 0.0001 | **** |
| FFA + TNF | LIRNAC + L-carnitine | 10 | 10 | 0.78 | 0.13 | 3 | 0.0241 | * |
| FFA + TNF | HMDB* | 1 | 0 | 1.00 | 0.14 | 3 | | |
| No FFA No TNF | HMDB* | 1 | 0 | 0.6 | 0.05 | 3 | 0.0004 | *** |

*HMDB: amino acid concentration based on the mean physiological concentrations in blood. The values are published in the Human Metabolome Database (HMDB).

### Example 2. Hepatocyte Model for NASH

Primary human hepatocytes (PHH) were used as a model to assess the ability of amino acids to influence fundamental aspects of NASH progression. Lipotoxicity is a major driver of hepatocellular injury due to dysregulated lipid metabolism, oxidative stress and mitochondrial dysfunctions. The PHH model was employed to assess the ability of amino acids to reduce disease phenotype by lowering lipotoxicity (lipid) and inflammation (MCP1 secretion), while promoting liver function by maintaining or increasing albumin secretion and maintaining or increasing urea production.

### Cell Seeding and Maintenance

PHH from two healthy human donors (Lonza, TRL) were seeded on day 0 at density of 6e04 cells in 96 well optical microplates (Thermofisher) in hepatocyte plating media (William's E medium, WEM) (Gibco) supplemented with 10% heat-inactivated FBS (Atlanta Bio), 2 mM Glutamax (Gibco), and 0.2% Primocin (InVivoGen) and incubated for 6 hours at 37° C., 5% CO₂. After 6 hours, cells were washed twice and incubated overnight at 37° C., 5% CO₂ with Hepatocytes defined medium (Corning) supplemented with 2 mM Glutamax (Gibco), and 1× Penicillin/Streptomycin. On day 1, cells were washed twice and incubated for 24 h in the hepatocyte culture media in the same conditions described above.

### Amino Acids Pre-Treatment

On day 2, cells were washed twice with DPBS 1× (Gibco) to remove excess amino acids and maintained in pretreatment media (1×HMDB, a defined custom amino acid concentration based on the mean physiological concentrations in blood, in WEM±supplemental amino acids):
a. Amino acid-free WEM (US Biologicals) supplemented with 11 mM Glucose (Sigma), 0.272 mM Sodium Pyruvate (Sigma), 1× P/S (Gibco) and containing 1×HMDB with no supplemental amino acids; or
b. The same media described above 1×HMDB WEM with supplemental amino acid and amino acid combination treatments at various concentrations.
   Cells were maintained in the defined media (a. & b.) for 24 hours at 37° C., 5% CO₂. The HMDB values are published in the Human Metabolome Database (Wishart D S, Tzur D, Knox C, et al., HMDB: the Human Metabolome Database. Nucleic Acids Res. 2007 January; 35 (Database issue):D521-6. 17202168; which is hereby incorporated by reference in its entirety).
   Co-Treatment with Free Fatty Acids and Amino Acids Combination

After 24 h pre-treatment, cells maintained in the pretreatment media±supplemental amino acids (section (a. & b.) described above) were exposed to free fatty acids (FFA) at 250 uM with a ratio of 2:1 (Oleate:Palmitate) supplemented with TNF-α (Thermofisher) at 1 ng/ml. Cells were incubated with the FFAs mixture (FFAs+TNF-α)±supplemental amino acids treatments for 24 hours at 37° C., 5% CO₂. After 24 hours incubation, media was removed for cytokine analysis and replaced by fresh media containing the same conditions described above (FFAs mixture±supplemental amino acids treatments). Cells were incubated for an additional 48 hours for a total of 72 hours of FFA and TNF-α stimulation. After 72 hours of incubation, media was removed for albumin and urea analysis and the cells were fixed for nuclei and lipid staining.

Cytokine Analysis after 24 h by ELISA

Human CCL2 (MCP-1) was measured by ELISA (Human CCK2/MCP-1 DuoSet ELISA, R&D Systems) at 1/20 dilution in 1× Reagent Diluent (Reagent Ancillary Kit 2, R&D Systems). MCP1 data in primary human hepatocytes treated with FFA+TNFα+supplemental amino acids was normalized to 1×HMDB WEM+FFA+TNFα baseline. Data is reported as median fold change from control, statistical analysis was done using t-Test.

Intracellular Lipid Accumulation Analysis after 72 h by Fluorescence Microscopy

After 72 hours, cells were washed twice in 100 ul PBS 1× (Gibco), fixed with 4% Paraformaldehyde, and washed twice with PBS 1× (100 ul). After fixation, lipids were stained with HCS LipidTOX Red Neutral (Thermofisher Scientific) diluted 1000× and nuclei were stained with Hoechst 3342 (Life Technologies) diluted to 4 ug/ml. The LipidTOX™ neutral lipid stain has an extremely high affinity for neutral lipid droplets that was detected by fluorescence microscopy using a high content imager (Molecular Devices). Data was normalized to the cell concentration determined by nuclei count. Images were analyzed and total lipid area was calculated using granularity module in MetaXpress 6 software. Data is reported as median fold change from control, statistical analysis was done using t-Test.

Albumin Analysis after 72 h by ELISA

Albumin secretion was measured as a functionality test in primary human hepatocytes. Albumin was measured by ELISA (Human Albumin ELISA Quantitation set, Bethyl Laboratories) at 1/200 dilution in the sample diluent (Bethyl Laboratories). Data is reported as median fold change from control, statistical analysis was done using t-Test.

Urea Analysis after 72 h by Urea Nitrogen (BUN) Colorimetric Assay

Urea was measured as an indicator of hepatocytes functionality by urea nitrogen direct assay (StanBio). Cell supernatant was added to the BUN color reagent and BUN acid reagent at 1(color):2(acid) ratio. Incubate the cell supernatant with BUN reagent for 12 min at 100° C. then for 5 min at 4° C. Read absorbance at 520 nm. Data was normalized to the control. Data is reported as median fold change from control, statistical analysis was done using t-Test.

### Results (Hepatocyte Donor 1)

The ability of single amino acids and combinations of amino acids to impact relevant disease phenotypes (MCP1, lipid) and functionality markers (albumin, urea) were measured and compared in the PHH model using healthy human hepatocyte donor 1.

MCP1 secretion data in Table 7, shows that treatment with LIRQNacSCar, LIRNacSCar, LIRNacCar, RQNac and Nac reduced MCP1 secretion. Treatment with single amino acids (L, I, V, R, Q, S, Car) and with combinations (LIV, LIVRQ, RQ) did not reduce MCP1 secretion. LIRQNacSCar deceased MCP1 secretion greater than any other single amino acid or combination.

Lipid accumulation data in Table 7, shows that treatment with LIRQNacSCar, LIRNacCar, LIRNacSCar, L, I, V, LI, LIV, Car, S and Q reduced lipid accumulation. RQNac, LIVRQ, RQ, R and Nac did not reduce lipid accumulation.

Albumin secretion data in Table 7 shows that treatment with LIRNacCar and LIRNacSCar, R, Car, and I did not reduce albumin secretion. Treatment with LIRQNacSCar, Nac and RQNac slightly decreased albumin secretion. Treatment with L, LI, V, RQ, Q, S, LIV, and LIVRQ decreased albumin production at a higher level than the previously described amino acids.

Urea production data in Table 7, shows that LIRQNacSCar, LIRNacCar, LIRNacSCar, R, RQ, RQNac, LIVRQ induced urea production. No changes in urea production were observed with Car, I, Nac, Q, S and L treatments.

### Summary of the Results (Hepatocyte Donor 1)

As described, an ideal treatment is one that addresses the multifactorial pathology of NASH as represented by PHH by reducing disease phenotypes (MCP1, Lipid) and promoting liver function (maintaining or increasing albumin, and maintaining or increasing urea). The ability of single amino acids and combinations to simultaneously impact these phenotypes was measured by a META-rank score (Table 7). META-rank score is a composite measure that considers the optimal impact on all 4 phenotypes (e.g. decrease MCP1, decrease lipid, maintain or increase albumin, maintain or increase urea) in the PHH model. An optimal amino acid or combination treatment (i.e. treatment that has the desired effect on all measures) has a lower score than a sub-optimal treatment (i.e. treatment has an undesirable effect on all measures). Based on META-rank, the combinations LIRQNacSCar, LIRNacCar and LIRNacSCar were the most optimal treatments.

TABLE 7
Changes MCP1, lipid, albumin and urea level in hepatocytes (donor 1) upon
administration of single amino compared to amino acids compositions LIRQNacSCar,
LIRNacCar and LIRNacSCar

| | AA | | | | | | | | | |
|---|---|---|---|---|---|---|---|---|---|---|
| Amino | Conc. (x) | | | | | | | | | |
| Acid | Nac/Car | MCP1 | | Lipid | | Albumin | | Urea | | Meta- |
| Supplement | (mM) | Median | p-value | Median | p-value | Median | p-value | Median | p-value | Rank |
| LIRQNacSCar | 20X | −2.431 | 0.0043 | −0.399 | 5.92E−19 | −0.481 | 0.0072 | 1.985 | 0.0002 | 5.75 |
| | 5 mM | | | | | | | | | |
| LIRNacCar | 20X | −1.398 | 0.0512 | −0.381 | 3.78E−23 | 0.255 | 0.2909 | 1.332 | 0.0003 | 6.25 |
| | 5 mM | | | | | | | | | |
| LIRNacSCar | 25X | −1.187 | 0.0034 | −0.377 | 1.54E−22 | 0.265 | 0.0383 | 1.375 | 0.0001 | 6.5 |
| | 5 mM | | | | | | | | | |
| R | 25X | 0 | 0.7152 | −0.007 | 0.142979 | 0.041 | 0.2446 | 1.836 | 0.0003 | 9.5 |
| RQNac | 20X | −2.337 | 0.0011 | 0.285 | 8.35E−24 | −0.276 | 0.0158 | 1.881 | 0.0002 | 9.5 |
| Car | 5 mM | −0.23 | 0.13 | −0.366 | 6.27E−26 | −0.064 | 0.0239 | −0.087 | 0.9191 | 11.25 |
| I | 20X | 0.074 | 0.5048 | −0.105 | 1.31E−06 | 0.051 | 0.0197 | 0.033 | 0.3767 | 12 |
| Nac | 5 mM | −1.316 | 0.0002 | −0.013 | 0.2023 | −0.28 | 0.0732 | −0.012 | 0.71 | 12 |
| Q | 20X | −0.235 | 0.0522 | −0.588 | 2.49E−24 | −2.158 | 0.0056 | 0.064 | 0.6054 | 12.75 |
| S | 25X | −0.082 | 0.3627 | −0.178 | 4.76E−15 | −0.714 | 0.0047 | 0.118 | 0.0209 | 12.75 |
| L | 20X | 0.418 | 0.1822 | −0.725 | 8.16E−35 | −0.649 | 0.0117 | −0.235 | 0.0051 | 13.5 |
| LIV | 20X | −0.097 | 0.3491 | −0.894 | 8.97E−28 | −1.376 | 0.0005 | −1.097 | 0.0003 | 13.5 |
| LIVRQ | 20X | 0.025 | 0.9791 | 0.09 | 0.3137 | −1.089 | 0.0043 | 1.915 | 0.0001 | 13.5 |
| LI | 20X | 0.007 | 0.5986 | −0.761 | 6.53E−33 | −1.059 | 0.0075 | −0.598 | 0.0025 | 13.75 |
| V | 20X | 0.219 | 0.1714 | −0.685 | 1.17E−23 | −0.9 | 0.0129 | −0.341 | 0.0051 | 14.5 |
| RQ | 20X | 0.756 | 0.0734 | 0.221 | 3.92E−11 | −0.868 | 0.0045 | 1.747 | 0.0006 | 14.75 |

*X correspond to the amino acid concentration values relative to the mean physiological concentrations in blood HMDB. The values are published in the Human Metabolome Database (HMDB).

### Results 2 (Hepatocyte Donor 2)

The ability of single amino acids and combinations of amino acids to impact relevant disease phenotypes (MCP1, lipid) and functionality markers (albumin, urea) were measured and compared in the PHH model using healthy human hepatocyte donor 2.

MCP1 secretion data in Table 8, shows that the combinations RQNac and LIRQ[2.5]NacCar reduced MCP1 secretion at the highest level followed by Nac, then LIRQ[2.5]NacSCar, NacCarS. The combinations LIRNacCar and LIRNacSCar also reduced MCP1 but at a lower level than the previously described combinations. LI, S, LIV and Car did not affect MCP1 secretion.

Lipid accumulation data in Table 8, shows that NacCarS and the following combinations LIRNacCar, LIRQ[2.5]NacCar, LIRNacSCar and LIRQ[2.5]NacSCar, reduced lipid accumulation. S, LI, Nac, Car and LIV reduced lipid accumulation but less than the previously described combinations. RQNac slightly increased lipid accumulation.

Albumin secretion data in Table 8, shows that Nac induce albumin secretion. LIRNacCar didn't affect albumin secretion. The combinations LIRQ[2.5]NacSCar, LIRQ[2.5]NacCar, LI, LIRNacSCar and NacCarS slightly reduced albumin secretion. Car, S, LIV, and RQNac reduced albumin secretion with RQNac reduced in albumin secretion the strongest.

Urea production data in Table 8, shows that RQNac, LIRQ[2.5]NacCar, LIRQ[2.5]NacSCar, LIRNacSCar, LIRNacCar significantly increased urea production. NacCarS, S, LI, LIV and Nac slightly induce urea production compared to the previously cited combinations. Car slightly reduced urea production.

TABLE 8
Changes in MCP1, lipid, albumin and urea level in hepatocytes (donor 2) upon
administration of various amino acid compositions

| | AA | | | | | | | | | |
|---|---|---|---|---|---|---|---|---|---|---|
| | Conc. | | | | | | | | | |
| Amino | (x)* | | | | | | | | | |
| Acid | Nac/Car | MCP1 | | Lipid | | Albumin | | Urea | | Meta- |
| Supplement | (mM) | Median | p-value | Median | p-value | Median | p-value | Median | p-value | Rank |
| LIRQ [2.5] | 30X | −5.391 | 0.002 | −1.864 | 9.37E−44 | −0.356 | 0.007 | 1.827 | 5.64E−05 | 9.75 |
| NacCar** | 7.5 mM | | | | | | | | | |
| LIRNacCar | 30X | −3.777 | 3.62E−05 | −2.432 | 1.29E−49 | 0.05 | 0.2599 | 1.641 | 2.41E−07 | 12 |
| | 7.5 mM | | | | | | | | | |
| LIRQ [2.5] | 30X | −4.795 | 0.0045 | −1.673 | 4.09E−48 | −0.311 | 0.0006 | 1.759 | 2.09E−05 | 12.75 |
| NacSCar** | 7.5 mM | | | | | | | | | |
| Nac | 7.5 mM | −5.235 | 0.0012 | −0.359 | 0.0022 | 0.372 | 0.0099 | 0.035 | 0.2141 | 13.5 |
| NacCarS | 30X | −4.705 | 0.0043 | −2.81 | 4.60E−44 | −0.386 | 0.0138 | 0.356 | 0.0074 | 13.75 |
| | 7.5 mM | | | | | | | | | |
| RQNac | 30X | −5.418 | 0.0017 | 0.149 | 4.99E−06 | −1.503 | 0.0062 | 2.48 | 7.40E−05 | 15.25 |
| | 7.5 mM | | | | | | | | | |
| LIRNacSCar | 30X | −3.279 | 0.0143 | −1.383 | 2.86E−05 | −0.386 | 0.0248 | 1.617 | 0.0007 | 17.75 |
| | 7.5 mM | | | | | | | | | |
| S | 30X | 0.1 | 0.1958 | −0.42 | 0.012 | −0.65 | 0.0211 | 0.297 | 0.0108 | 21.5 |
| Car | 7.5 mM | −0.275 | 0.2024 | −0.32 | 0.0155 | −0.504 | 0.0048 | −0.312 | 0.0003 | 22.25 |
| LI | 30X | 0.052 | 0.1285 | −0.248 | 4.71E−09 | −0.388 | 0.0131 | 0.168 | 0.0005 | 22.5 |
| LIV | 30X | −0.16 | 0.2324 | −0.303 | 6.11E−17 | −0.986 | 0.0006 | 0.103 | 0.0302 | 22.75 |

*X correspond to the amino acid concentration values relative to the mean physiological concentrations in blood HMDB. The values are published in the Human Metabolome Database (HMDB).
**Q was used at 2.5X concentration relative to 1X HMDB value in these combinations.

### Summary of the Results (Hepatocyte Donor 2):

As described, an ideal treatment is one that addresses the multifactorial pathology of NASH as represented by PHH by reducing disease phenotypes (MCP1, Lipid) and supporting hepatocyte function (maintaining or increasing albumin, and maintaining or increasing urea). The ability of single amino acids and combinations to simultaneously impact these phenotypes was measured by a META-rank score (Table 8). META-rank score is a composite measure that considers the optimal impact on all 4 phenotypes (e.g. decrease MCP1, decrease lipid, maintain albumin, maintain or increase urea) in the PHH model. An optimal amino acid or combination treatment (i.e. treatment that has the desired effect on all measures) has a lower score than a sub-optimal treatment (i.e. treatment has an undesirable effect on all measures). Based on META-rank, the combinations LIRQ[2.5]NacCar, LIRQ[2.5]NacSCar followed by LIRNacCar were the most optimal treatments. Nac and NacCarS were equally ranked in their ability to impact NASH followed by RQNac and LIRNacSCar. LI, S, Car and LIV had the least impact on affecting the disease phenotypes compared to the previously described amino acids combinations.

### Example 3. Hepatocyte Model for Measuring Triglyceride Level

Triglyceride (TG) accumulation in the cytoplasm of hepatocytes is a hallmark of NAFLD/NASH. The ability of amino acids to reduce triglyceride level was assessed using human primary hepatocytes (Lonza, TRL).

### Cell Seeding and Maintenance

Primary hepatocytes from two healthy human donors (hepatocyte donor 2 and 3) were seeded on day 0 at density of 3.5e05 cells in 24 well collagen coated plate (Corning) in hepatocyte plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO₂. After 6 hours, cells were washed twice and incubated overnight at 37° C., 5% CO₂. On day 1, cells were washed twice and incubated at 37° C., 5% CO₂ for 24 h with hepatocytes defined medium (Corning) supplemented with 2 mM Glutamax (Gibco), and 1× Penicillin/Streptomycin (P/S).

### Amino Acids Pre-Treatment

On day 2, cells were washed twice with DPBS 1× (Gibco) and maintained in:
a. Amino acid-free WEM (US Biologicals) supplemented with 11 mM Glucose (Sigma), 0.272 mM Sodium Pyruvate (Sigma), 1× P/S (Gibco) and containing a defined custom amino acid concentration based on the mean physiological concentrations in blood (1× HMDB WEM (−) supplemental amino acids); or
b. The same media described above 1×HMDB WEM (+) supplemental amino acids treatment dose at a 30× of HMDB amino acid concentration.
   Cells were maintained in the defined amino acids media (a. & b.) for 24 hours at 37° C., 5% CO₂.
   Co-Treatment with Free Fatty Acids and Various Amino Acids Supplemental Treatments

After 24 h pre-treatment, cells maintained in the same media (a. & b.) described above 1×HMDB±supplemental amino acids were now exposed to free fatty acids (FFA) at 250 uM with a ratio of 2:1 (Oleate:Palmitate) supplemented with TNF-α (Thermofisher) at 1 ng/ml or vehicle. Cells with the FFAs+TNFα mixture±amino acids treatments were incubated for 72 hours at 37° C., 5% CO₂ with a media change after the first 24 hours.

Intracellular Triglyceride Level after 72 h

After 72 hours, cells were washed once with cold PBS 1× (Gibco), scrapped off and collected into 75 ul of standard diluent (Cayman). Collected cells were vortexed for 1 min at a 2500 rpm speed followed by two times sonication (Elmasonic sonicator) for 1 min each. Supernatants were collected for triglyceride analysis after centrifugation at 1000 rpm for 10 min at 4° C. Intracellular triglyceride level was assessed using triglyceride colorimetric assay kit (Cayman) and following manufacturer's recommendations. Data was normalized to the total amount of protein using Bicinchoninic Acid Protein Assay Kit (Sigma). Data is reported as median fold change from FFAs+TNFα condition, statistical analysis was done using t-Test.

### Hepatocyte Triglyceride Results

Table 9 shows the fold change in triglyceride level in primary human hepatocytes from two healthy donors treated with FFAs+TNFα and supplemented with amino acids or amino acid combinations. The treatment with LIRNacSCar, LIRNacCar and LIRQNacSCar decreases triglycerides to the vehicle level (No FFA, No TNF in 1×HMDB). S alone didn't have an impact on triglycerides, however when combined with LIRNacCar triglyceride level was reduced further (LIRNacSCar compared to LIRNacCar). Car, LI and Nac alone reduces triglyceride level but the effect was less pronounced than the combinations described previously.

TABLE 9
Changes in triglyceride level in hepatocytes upon
administration of amino acid compositions

| | | | | Triglyceride Level | |
|---|---|---|---|---|---|
| | | AA | | relative to the | |
| | Amino Acid | Conc | Nac/Car | baseline FFA + TNF | |
| Treatment | Supplement | (X) | (mM) | Median | p-Value |
| FFA + TNF | HMDB* | 1 | 0 | −0.303 | 0.1991 |
| FFA + TNF | Car | 1 | 7.5 | −0.809 | 0.0222 |
| FFA + TNF | LI | 30 | 0 | −1.312 | 0.0029 |
| FFA + TNF | LIRNacCar | 30 | 7.5/7.5 | −1.646 | 0.0132 |
| FFA + TNF | LIRNacSCar | 30 | 7.5/7.5 | −1.879 | 0.0074 |
| FFA + TNF | LIRQNacSCar | 30 | 7.5/7.5 | −1.634 | 0.0010 |
| FFA + TNF | Nac | 1 | 7.5 | −1.172 | 0.0087 |
| FFA + TNF | S | 1 | 0 | −0.176 | 0.1817 |
| No FFA | HMDB* | 1 | 0 | −1.428 | 0.0008 |
| No TNF | | | | | |

*HMDB: amino acid concentration based on the mean physiological concentrations in blood. The values are published in the Human Metabolome Database (HMDB).

### Example 4. TGFβ Stimulation of Hepatic Stellate Cells to Model Fibrosis

Fibrosis is at the nexus of several biologic processes, such as metabolic dysregulation, inflammation, and cell death. Lipid accumulation in hepatocytes and chronic inflammation induce fibrogenic activation of hepatic stellate cells (Wobser H, et al., Cell Res. 2009, which is hereby incorporated by reference in its entirety).

In response to liver injury hepatic stellate cells become activated, proliferative, increase production of αSMA, secretion of type I and III collagens and specific MMP and TIMP proteins. Primary human hepatic stellate cells (HSC) were selected as a model of activated hepatic stellate cells and used to test whether specific amino acid compositions would reduce fibrogenic markers induced by TGFβ treatment, specifically alpha-SMA, Procollagen 3, and rate of EdU incorporation.

Primary human hepatic stellate cells were obtained from Samsara Sciences. Cells from three different donors were grown in Complete HSC Medium to ˜80% confluence in T75 or T150 flasks below passage 10 were seeded into sterile, collagen I coated, 96-well optical plastic microplates (ThermoScientific) and incubated overnight at 37° C., 5% CO₂ in a humidified incubator in DMEM with 2% Fetal Bovine Serum and 1% Antimycotic. After the overnight incubation, plates are washed and incubated in pretreatment medium (1×HMDB amino acid DMEM+1% Antimycotic, 10 mM HEPES, ±supplemental amino acids treatment dose at a 30× of the HMDB amino acid concentration) for 10.5 hours. After 10.5 hour of incubation, the same pretreatment medium, now supplemented with 3 ng/mL TGFβ1, was applied to the cells and incubated for 24 hours at 37° C., 5% CO2. After 24 hours of stimulus, supernatant was removed and used to measure Procollagen III and cells were then washed and fixed with 4% paraformaldehyde for further staining.

Procollagen III, an important biomarker of fibrosis was measured by ELISA (Human PCIII Elisa Kit, G-Biosciences) following the manufacturer's recommendations. Fixed cells with 4% paraformaldehyde were permeabilized with 0.1% Triton X-100 then immunostained for alpha SMA and labeled for EdU incorporation. To measure alpha SMA, primary antibody Alpha-Smooth Muscle Actin Monoclonal Antibody (1A4) isotype IgG2a, eBioscience (Thermofisher) was used in conjugation with the corresponding secondary antibody Goat anti-Mouse IgG2a Cross-Adsorbed Secondary Antibody, Alexa Fluor 647. EdU was labeled using the Click-iT™ EdU Alexa Fluor™ 555 HCS Assay (Invitrogen) according to the manufacturer's instructions.

Nuclei were labeled with Hoechst 33342, a cell permeable DNA binding dye. Cells were imaged using ImageXpress micro confocal high content imager (Molecular Devices). Alpha SMA labeled with Alexa Fluor 647 was detected in the Cy3 channel. EdU labeled with Alexa Fluor™ 555 was detected in the Texas Red channel. Nuclei labeled with Hoechst 33342 were detected in the DAPI channel. Image analysis was performed using MetaXpress version 6.2.3.733 (Molecular Devices).

### Results

### Procollagen III Secretion

Procollagen III (ProC3) is a major noninvasive fibrosis biomarker in NASH. Table 10 shows the fold change in procollagen III in primary human hepatic stellate cells from three different healthy donors normalized to their respective baseline amino acid conditions. LIRNacCar and LIRQNacSCar significantly decreased procollagen III secretion and had the highest impact on procollagen III reduction followed by Nac, RQNac RQ, Q and LIVRQ which decreased procollagen III secretion but at a lower extent than LIRNacCar and LIRQNacSCar. V, S, LI, L R, Car, I and LIV, had no impact on procollagen III secretion.

### HSC Proliferation Measured by EdU Incorporation

Proliferation is a key characteristic of HSC activation. EdU labeled cells were analyzed and the number of proliferating cells, defined as those nuclei that were positive for EdU labeling (EdU+) and the total nuclei count were determined for each condition. The percentage EdU positive cells (% EdU+) was determined as the number of EdU positive nuclei divided by the total number of nuclei for each well. Fold change in % EdU+ cells were calculated relative to the baseline amino acid condition stimulated with 3 ng/mL TGFβ1.

Table 10 shows the fold change in the percentage of actively proliferating EdU positive cells, relative to the baseline condition in primary human hepatic stellate cells. LIRQNacSCar reduced the percentage of actively proliferating EdU positive cells relative to 1×HMDB stimulated with 3 ng/mL TGFβ1. The remaining combinations including LIRNacCar as well as individual amino acids reduced the percent of actively proliferating EdU positive cells but less than LIRQNacSCar.

### HSC Activation Measured by Alpha SMA

Fixed cells immunostained for alpha smooth muscle actin (αSMA) were analyzed and stained area was normalized to total nuclei count. Fold change were calculated relative to the baseline amino acid (1×HMDB) condition stimulated with 3 ng/mL TGFβ1.

Table 10 shows that LIRQNacSCar reduced the alpha SMA stained area at a high level along with LI and Nac, followed by R, LIRNacCar, Car and LIVRQ.

TABLE 10
Effect of amino acid compositions on markers of fibrosis (proC3, aSMA, edu
incorporation) in HSCs

| | AA | | | | | | | |
|---|---|---|---|---|---|---|---|---|
| | Conc. | | | | | | | |
| Amino | (X)* | | | | | | | |
| Acid | Nac/Car | Procollagen3 | | alpha SMA | | edU incorporation | | |
| Supplement | (mM) | Median | p-value | Median | p-value | Median | p-value | Meta-Rank |
| Car | 5X | −0.028 | 0.6538 | −0.222 | 0 | −0.251 | 0.0013 | 14.333 |
| I | 20X | −0.001 | 0.0689 | −0.077 | 0.014 | −0.25 | 0.0063 | 19 |
| L | 20X | −0.073 | 0.5584 | −0.149 | 0.0001 | −0.095 | 0.2986 | 17.333 |
| LI | 20X | −0.087 | 0.5524 | −0.35 | 0 | −0.195 | 0.0145 | 12 |
| LIRNacCar | 20X | −1.111 | 0.0006 | −0.235 | 0 | −0.882 | 0 | 5.667 |
| | 5 mM | | | | | | | |
| LIRQNacSCar | 20X | −1.098 | 0.0002 | −0.302 | 0 | −1.993 | 0 | 3 |
| | 5 mM | | | | | | | |
| LIV | 20X | 0.038 | 0.903 | −0.075 | 0 | −0.118 | 0.0148 | 20.333 |
| LIVRQ | 20X | −0.248 | 0.0013 | −0.221 | 0 | −1.074 | 0.0001 | 8.333 |
| Nac | 5 mM | −0.765 | 0.000002 | −0.332 | 0 | −0.475 | 0.0001 | 7.667 |
| Q | 20X | −0.32 | 0.0637 | −0.111 | 0.3991 | −0.965 | 0 | 11.667 |
| R | 25X | −0.033 | 0.571 | −0.253 | 0 | −0.601 | 0 | 11.667 |
| RQ | 20X | −0.472 | 0.0074 | −0.045 | 0.0057 | −0.825 | 0.0001 | 13.667 |
| RQNac | 20X | −0.618 | 0.0005 | −0.079 | 0 | −0.981 | 0 | 10.667 |
| | 5 mM | | | | | | | |
| S | 25X | −0.162 | 0.0558 | −0.175 | 0.0009 | −0.321 | 0.0168 | 14 |
| V | 20X | −0.242 | 0.1082 | −0.151 | 0 | −0.372 | 0.0147 | 13.667 |

*X correspond to the amino acid concentration values relative to the mean physiological concentrations in blood HMDB. The values are published in the Human Metabolome Database (HMDB).

### Summary of HSC Results:

As described, an ideal treatment is one that addresses the multifactorial pathology of NASH as represented by HSC by reducing disease phenotypes (aSMA, ProC3, and EdU). The ability of single amino acids and combinations to simultaneously impact these phenotypes was measured by a META-rank score (Table 10). META-rank score is a composite measure that considers the optimal impact on all 3 measures (e.g. decrease in each of aSMA, ProC3, and EdU) in the HSC model. An optimal amino acid or combination treatment (i.e. treatment that has the desired effect on all measures) has a lower score than a sub-optimal treatment (i.e. treatment has an undesirable effect on all measures). Based on META-rank, amino acid compositions LIRQNacSCar showed highest impact on fibrosis markers followed by LIRNacCar.

### Example 5. Primary Human Macrophages: Metabolic Switch Toward a Less Inflammatory Phenotype

It is well established that severe inflammation can drive progression of liver diseases, such as cirrhosis, fibrosis, and hepatocellular carcinoma. Characteristic response to liver injury involves increased numbers of “activated” (M1) macrophages at sites of injury along with enhanced production of cytotoxic and proinflammatory mediators. Recent studies have suggested that M2 macrophages play an essential role in NASH by suppressing inflammation and initiating wound healing, and that abnormalities in macrophage activation state and reprogramming from (M2) immunosuppressive phenotype to (M1) activities can lead to an exaggerated response to liver injury and to the development of fibrosis or cancer. Currently, macrophages are potential targets for therapeutic options that aim to reduce the burden inflammation in NASH.

### Primary Human M1 Macrophages: Pro-Inflammatory Cytokine Secretion

Primary human PMBC derived macrophages were seeded on day 0 at 3.0E4 cells per well in 96-well microplates (ThermoFisher) in Dulbecco's Modified Eagle Medium (DMEM) (Gibco) supplemented with penicillin-streptomycin (Hyclone) and 10% heat inactivated fetal bovine serum (HI-FBS) (Atlanta Bio) and incubated overnight at 37° C., 5% CO2. On day 1, cells were washed once with 150 uL per well DPBS (Gibco) and treated with 75 uL of:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES, 0.2% primocin (InVivoGen); or
b. The same media described above with supplemental amino acid and amino acid combination treatments at various concentrations.

On day 2, cells were treated with 75 uL of the same media described above supplemented with 0.30 ng/mL lipopolysaccharide (LPS) (Sigma) for a final concentration of 0.15 ng/mL LPS. Control wells were treated with 1 uM BX-795 (Tocis), 1 uM TAK242 (Sigma), 0.15 ng/mL LPS, or phosphate buffered saline (PBS).

On day 3, the supernatant was collected and immediately frozen in −80° C. freezer. Cells were washed once with 150 uL DPBS and viability was assessed using the WST-8 Cell Proliferation Cytotoxicity Assay (Dojindo). Following the assay, cells were washed twice with 150 uL PBS and fixed with 4% paraformaldehyde for 5 min followed by two additional washes with 150 uL PBS. Protein levels in supernatant samples were analyzed by ELISA for IL-6 and TNFa using commercially available kits (R&D Systems) according to manufacturer-supplied protocols. Results are shown in Table 11 below.

TABLE 11
Effect of amino acids and amino acid compositions on
M1 macrophage IL6 and TNFa secretion

| | AA Conc. | | | | | |
|---|---|---|---|---|---|---|
| | (X)* | | | | | |
| Amino Acid | Nac/Car | IL-6 | | TNFa | | Meta- |
| Supplement | (mM) | Median | P-Value | Median | P-Value | Rank |
| Car | 5 mM | −0.136 | 0.0142 | −0.539 | <0.0001 | 10 |
| I | 20X | −0.101 | 0.0762 | 0.132 | 0.2097 | 14 |
| L | 20X | −0.019 | 0.6235 | −0.029 | 0.6802 | 13 |
| LIRNacCar | 20X/ | −1.233 | <0.0001 | −2.242 | <0.0001 | 1.5 |
| | 5 mM | | | | | |
| LIRNacSCar | 20X/ | −1.120 | <0.0001 | −2.090 | <0.0001 | 3.5 |
| | 5 mM | | | | | |
| LIRQNacSCar | 20X/ | −0.728 | 0.0005 | −1.886 | <0.0001 | 6 |
| | 5 mM | | | | | |
| LIV | 20X | 0.055 | 0.1705 | 0.437 | 0.0040 | 16 |
| LIVRQ | 20X | 0.609 | 0.0069 | 0.466 | 0.0001 | 19 |
| Nac | 5 mM | −1.442 | 0.0005 | −1.631 | <0.0001 | 3.5 |
| Q | 20X | 0.480 | 0.0023 | 0.446 | 0.0840 | 18 |
| R | 25X | 0.040 | 0.6797 | −0.060 | 0.5401 | 12.5 |
| RQNac | 20X/ | −0.876 | 0.0033 | −1.509 | 0.0006 | 6.5 |
| | 5 mM | | | | | |
| S | 25X | 0.224 | 0.1715 | 0.005 | 0.2229 | 15.5 |
| V | 20X | −0.369 | 0.0005 | −0.011 | 0.1140 | 12 |

Table 11 shows the median fold change in IL-6 and TNF-alpha secretion from LPS treated control. The ability of single amino acids and combinations to simultaneously impact the studied phenotypes (IL-6 and TNFa) was measured by a META-rank score. META-rank score is a composite measure that considers the optimal impact on all phenotypes tested in the M1 macrophage model. Compositions comprising LIRNacCar, including LIRQNacSCar and LIRNacSCar, demonstrated robust reduction of LPS-stimulated inflammatory cytokine secretion, when compared to other amino acid combinations. Nac and RQNac, and to a lesser extent Car, also decreased LPS-stimulated IL-6 and TNFa secretion. Reduction of LPS-stimulated secretion of IL-6 and TNFa indicates an anti-inflammatory effect of compositions.

### Primary Human M2 Macrophages: Anti-Inflammatory Chemokine Secretion

On day 0, primary human PBMC derived macrophages were seeded in Dulbecco's Modified Eagle Medium (DMEM) (Gibco) supplemented with penicillin-streptomycin (Hyclone) and 10% heat inactivated fetal bovine serum (HI-FBS) (Atlanta Bio) into 96-well microplates (ThermoFisher) and incubated overnight at 37° C., 5% CO2. On day 1, cells were washed once with DPBS and then treated with:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES, penicillin-streptomycin; or
b. The same medium described above with one amino acid or amino acid combination at various concentrations

On day 2, cells were treated with the same mediums described above supplemented with recombinant human interleukin-4 (1 ng/ml of IL-4; Peprotech). Control wells were treated with tofacitinib (Tocris) and IL-4, PBS with IL-4, or phosphate buffered saline (PBS) alone.

On day 3, the supernatant was collected and immediately frozen in −80° C. freezer. Cells were washed once with DPBS and viability was assessed using the WST-8 Cell Proliferation Cytotoxicity Assay (Dojindo) according to manufacturer-supplied protocol. Following the assay, cells were washed twice with PBS and fixed with 4% paraformaldehyde. Protein levels in supernatant samples were analyzed by ELISA for CCL18 using commercially available kits (R&D Systems) according to manufacturer-supplied protocols.

TABLE 12
Effect of amino add compositions on secreted
CCL18 levels in M2 macrophages

CCL18

| | Median Fold | |
|---|---|---|
| | Change from | |
| Supplement | Vehicle (log2) | P-Value |
| Car (5 mM) | 0.335 | 0.0260 |
| I (20X) | 0.125 | 0.0399 |
| L (20X) | 0.749 | <0.0001 |
| LIRNac (20X/5 mM Nac/Car) | 1.398 | 0.0004 |
| LIRNacCar (20X/5 mM Nac/Car) | 1.953 | 0.0069 |
| LIRNacSCar (20X/5 mM Nac/Car) | 1.716 | 0.0031 |
| LIRQNacSCar (20X/5 mM Nac/Car) | 2.199 | 0.0044 |
| LIV(20X) | 0.547 | 0.0337 |
| LIVRQ (20X) | 1.950 | 0.0002 |
| Nac (5 mM) | 0.387 | 0.0068 |
| Q (20X) | 0.979 | <0.0001 |
| R (25X) | 0.111 | 0.0231 |
| RQNac (20X) | 1.568 | <0.0001 |
| S (25X) | 0.181 | 0.0002 |
| V (20X) | 0.358 | 0.0300 |

Table 12 shows the median fold change in CCL18 secretion from IL-4 treated control. Induction of CCL18 cytokine secretion indicates an anti-inflammatory effect mediated by a shift of macrophages to a more anti-inflammatory phenotype. Compositions comprising LIRNacCar, including LIRQNacSCar, demonstrated robust anti-inflammatory effect when compared to single amino acids or other amino acid combinations, with LIRQNacSCar demonstrating superior anti-inflammatory effect. LIRNacCar and LIRNacSCar, as well as LIVRQ also increased CCL18 secretion.

### Example 6. ATP Production Rate as Indicator of Induction of Metabolic Switch

Activation of macrophages with proinflammatory stimuli induces a metabolic switch from oxidative phosphorylation to glycolysis. This activation leads to increases in glycolysis, lactate production and glycolytic ATP levels, as well as reduction in mitochondrial ATP (with increases in TCA cycle intermediates succinate and citrate) and increases in inflammatory cytokines and reactive oxygen species. Such metabolic changes in macrophages can contribute to NAFLD progression. Effects of the amino acid and amino acid combination treatments on M1 macrophage metabolism were assessed using a real-time ATP rate assay.

Primary human PMBC derived macrophages were seeded on day 0 at 2.0E4 cells per well in a Seahorse X96 Cell Culture Microplate V3-PS TC-Treated plate (Agilent) coated with 0.1 mg/mL Poly-D-Lysine (Trevigen) in Dulbecco's Modified Eagle Medium (DMEM) (Gibco) supplemented with penicillin-streptomycin (Gibco) and 10% heat inactivated fetal bovine serum (HI-FBS) (Atlanta Bio) and incubated overnight at 37° C., 5% CO2. On day 1, cells were washed once with 150 uL per well DPBS (Gibco) and treated with 100 uL of:
a. Amino acid free DMEM (US Biologicals) containing a defined custom amino acid concentration based on the mean physiological concentrations in blood based on values published in the Human Metabolome Database (HMDB), with 6 mM glucose, 1 mM sodium pyruvate, 10 mM HEPES (Sigma), and penicillin-streptomycin (Gibco); or
b. The same media described above with supplemental amino acid and amino acid combination treatments at various concentrations.

On day 2, cells were treated with 100 uL of the same media described above supplemented with 0.15 ng/mL lipopolysaccharide (LPS) (Sigma). Control wells were treated with 0.15 ng/mL LPS, or phosphate buffered saline (PBS).

On day 3, the supernatant was collected and immediately frozen in a −80° C. freezer. Cells were analyzed for total ATP production rate, glycolytic ATP production rate, and mitochondrial ATP production rate using a commercially available kit (Agilent Seahorse XF Real-Time ATP Rate Assay Kit) according to manufacturer-supplied protocol on a Seahorse XFe instrument. A custom assay medium (amino acid free DMEM/F12 without phenol red or sodium bicarbonate (US Biologicals) containing a defined custom amino acid concentration based on the mean physiological concentrations in blood based on values published in the Human Metabolome Database (HMDB), with 10 mM XF glucose (Agilent), 1 mM XF pyruvate (Agilent), and 5 mM HEPES (Sigma) was used. Buffer factor for the custom assay medium was determined according to manufacturer-supplied protocol. Following the assay, cells were washed twice with PBS and fixed with 4% paraformaldehyde.

TABLE 13
Glycolytic, Mitochondrial, and Total ATP Rate Measurements in M1 macrophages

| | | Glycolytic | | Mitochondrial | | | |
|---|---|---|---|---|---|---|---|
| | | ATP Rate | | ATP Rate | | Total ATP Rate | |
| | | Median | | Median | | Median | |
| | | Fold | | Fold | | Fold | |
| | | Change | | Change | | Change | |
| | | from | | from | | from | |
| | | Vehicle | | Vehicle | | Vehicle | |
| Supplement | Conc. | (Log2) | P-Value | (Log2) | P-Value | (Log2) | P-Value |
| LIRQNacSCar | 30X/5 mM | −0.633 | <0.0001 | −0.017 | 0.890 | −0.1860 | 0.0122 |
| | Nac/Car* | | | | | | |
| Nac | 7.5 mM | −0.461 | <0.0001 | −0.196 | 0.003 | −0.2744 | 0.0002 |

Table 13 shows the ATP Rates in M1 macrophage following treatment with 30× LIRQNacSCar (including 7.5 mM Nac and Car) or 7.5 mM Nac normalized to LPS vehicle control. Treatment with LIRQNacSCar significantly decreased glycolytic ATP production. LIRQNacSCar treatment did not significantly alter mitochondrial ATP production. Due to the change in glycolytic ATP production rate, total ATP rate was also reduced. Treatment with Nac at 7.5 mM significantly decreased glycolytic, mitochondrial, and total ATP rate measurements. Decreases in glycolytic ATP production rates, without significant changes in mitochondrial ATP rate, suggest that treatment is moving cells towards a less glycolytic, less inflammatory metabolic state. Effect of LIRQNacSCar on glycolytic ATP production rates is significantly higher than effect of Nac (p=0.0354). This data demonstrates that LIRQNacSCar is better in reprogramming cell toward less glycolytic, less proinflammatory state. Results are reported as the median loge of the fold change relative to LPS vehicle control. P-values were determined by t-test.

### Example 7. Monitoring Homogeneity in Real Time Using NIR—Additional Combinations and Ribbon Blending

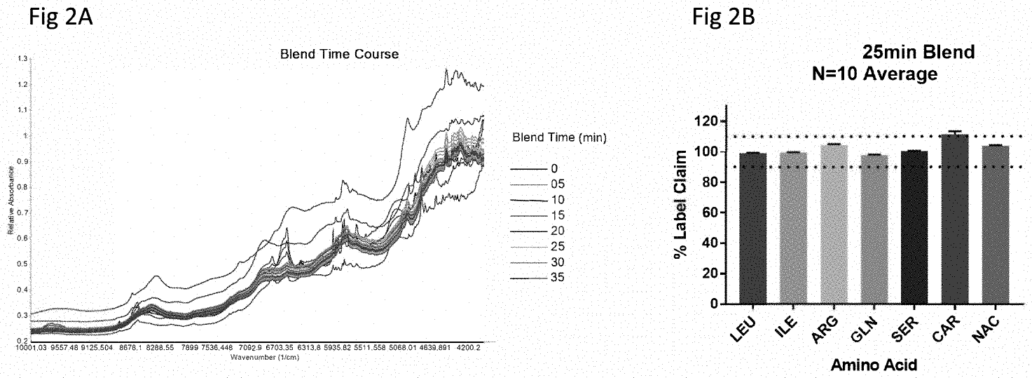

FIG. 2A shows multiple NIR spectra of samples taken during the ribbon blending of a second additional exemplary combination of amino acid entities (the exemplary combination of Table 2) at five-minute intervals. The collapse of the spectra at late time points to a single representative spectrum indicates the combination approaches blend uniformity. The histograms in FIG. 2B represents the average and their standard errors of the mean taken from HPLC chromatograms of ten randomly-selected and independent stick packs after 25 minutes of blending. The recovery data is expressed as a percent of label claim. The amino acid recovery values appear to conform to the 90-110 percent acceptance criteria (dotted line). The data when taken together indicated blend and content uniformity had been achieved.

These experiments demonstrate that the methods described herein may be used to achieve blend and content uniformity for additional combinations of amino acid entities and that a variety of blending techniques, including ribbon blending, are suitable for achieving uniformity.

### Example 8. Treatment of Non-Alcoholic Fatty Liver Disease (NAFLD) Subjects with an Amino Acid Composition

The study described herein features the administration of a composition including amino acids to subjects with non-alcoholic fatty liver disease (NAFLD). This is a 16-week, randomized, single-blind, placebo-controlled study to assess the safety and tolerability of the composition in subjects with NAFLD.

Safety and tolerability will be assessed by: reported clinical adverse events (AEs); physical examinations, including body weight; vital sign assessments; multiparametric magnetic resonance imaging (MRI) to characterize and quantify liver tissue (e.g. liver fat/inflammation) and body composition; electrocardiograms (ECGs); and clinical laboratory tests including changes in hematology, chemistry, lipid profiles, glucose homeostasis, and other blood markers of inflammation and fibrosis.

Subjects will be randomized to receive either the composition at a dosage of 20.3 g BID (n=30) or placebo at a dosage of 20.3 g BID (n=30). The placebo is calorie-, excipient-, and color-matched to the composition, but without any amino acids. A randomization scheme will be employed to allocate subjects evenly to the administration arms for both type 2 diabetes (T2D) subjects and non-T2D subjects.

Randomization will occur after eligibility. Assigned composition (LIRQNacCarS or placebo) will be shipped to the clinical site upon randomization of each subject. Once randomization has occurred, subjects will present to the study site on Day 1 (Baseline/Visit 2) for their baseline assessments. Study Day 1 is the beginning of the 16-week Administration Period. Subjects will return to the study site at Week 1 (Visit 3), Week 2 (Visit 4), Week 4 (Visit 5), Week 8 (Visit 6), Week 12 (Visit 7) and Week 16 (Visit 8) to receive their composition, to provide blood samples for biomarker and other laboratory testing, undergo liver imaging, and to complete other study safety assessments.

Maintenance of subjects' lifestyle regimen will be monitored via body weight assessments, recording changes in their diet, and recording changes in physical activity at every visit. During each study visit, subjects will meet with a study dietician or other qualified study staff (e.g., Investigator, trained study nurse, etc.). The study dietician (or other qualified staff) will review any dietary or activity changes from baseline. Subjects will also be reminded to continue to adhere to their usual baseline dietary and activity patterns.

The composition or placebo is provided as dry powder in stick packs. The specified number of stick packs (3 stick packs) at each administration should be reconstituted in 8 oz (˜240 ml) of water and consumed orally immediately after mixing. Daily administration of the composition should occur 30±5 minutes before meals twice daily (e.g., before breakfast and dinner or before lunch and dinner). The twice daily administrations should occur at least 4 hours apart. It is expected that subjects consume their total daily amount of the assigned composition (the composition or placebo).

### Example 9. Treatment of Non-Alcoholic Fatty Liver Disease (NAFLD) Adolescent Subjects with an Amino Acid Composition

The study described herein features the administration of a composition including amino acids to treat adolescent patients with non-alcoholic fatty liver disease (NAFLD). This is a randomized, single-blind, placebo-controlled study to assess the safety and tolerability of the composition in adolescent subjects with NAFLD.

Safety and tolerability will be assessed by: reported clinical adverse events (AEs); physical examinations, including body weight; vital sign assessments; multiparametric magnetic resonance imaging (MRI) to characterize and quantify liver tissue (e.g. liver fat/inflammation) and body composition; electrocardiograms (ECGs); and clinical laboratory tests including changes in hematology, chemistry, lipid profiles, glucose homeostasis, and other blood markers of inflammation and fibrosis. Liver structure and function will be assessed by: multiparametric magnetic resonance imaging (MRI) assessments of liver structure (fat content and inflammation changes) and blood tests of liver function, including markers of inflammation and fibrosis,

Male and female subjects between 12 and 17 years of age, inclusive, will be assessed. This is a randomized, placebo-controlled study conducted in 2 Parts: a mandatory 13-wk administration period (Part 1) and an optional extension period up to a maximum of 12 additional weeks (Part 2). The total duration of the study from Screening to the end of Part 2 follow-up is anticipated to be approximately 31 weeks. It is anticipated that there may be a total of approximately up to 12 study visits during the entire study (Screening, Parts 1 and 2).

In Part 1, following up to a 4-week screening period, eligible subjects will be randomized in a 2:1 ratio to receive either the composition or placebo for a 13-week administration period. Subjects will be centrally randomized with gender (male/female) as the stratification factor to the composition or placebo groups. Randomization will occur after eligibility is confirmed and prior to the Day 1 Visit. Composition amounts will be gradually escalated through the first week of study participation to assess tolerability issues to the food product during the initial week, and to enable subjects and their caregivers to get accustomed to the twice daily regimen. Subjects are anticipated to be on stable amounts of composition during Weeks 2-13, and safety and tolerability will be monitored throughout the study. All subjects will be provided diet and physical activity recommendations consistent with Guidance and Lifestyle Recommendations for Adolescents with NAFLD.

Following the mandatory Part 1 period, all subjects including those randomized to the placebo-arm in Part 1, will have an option to continue in the study in Part 2 for up to an additional maximum of 12 weeks on the composition. All subjects who opt to enter Part 2 will be administered the composition at the same amount and regimen as in Part 1 (i.e. up to 3 stick packs twice daily starting from Week 14) and will continue to be provided the standard lifestyle guidance. Subjects who choose not to participate in Part 2, will undergo a safety follow up visit approximately 2-weeks after Visit 6 per the procedures in Schedule of Assessments (SOA). There will be a 2-week follow up period after subjects complete Part 2.

The placebo is a balanced food product formulated as a dry powder that will be reconstituted with ˜6 oz (˜180 mL) of water to form an orange flavored drink that is color, taste, and calorie matched to the composition. The composition will be provided in dry powder form in stick packs, which are then mixed in ˜6 oz (˜180 mL) of water, and then consumed twice daily approximately 30 min (i.e., 30±5 minutes) before meals (e.g., before breakfast and dinner or before lunch and dinner, if breakfast is not a usual part of their daily routine) for the entire duration of the study.

### Part 1: Mandatory Administration Period (13 Weeks)

### Visit 1/Day 1 and Up to Week 1:

Eligible subjects will be randomized on Day 1 and will receive either placebo or the composition with instructions on how to prepare and consume the composition, including timing of administration. On Day 1, subjects will arrive to the study site following an overnight fast of approximately 8 hours. Prior to administration of the composition, subjects will undergo fasting blood draws and other assessments. The first administration will be at the study site supervised by the study staff. After administration of the composition, subjects will have one additional blood draw for plasma amino acid concentration approximately 1-2 hours after administration.

During the first week of administration in both the composition and placebo groups, subjects will follow the schedule: Days 1 to 3: 1 stick pack twice daily (total of 2 stick packs daily); and Days 4 to 7: 2 stick packs twice daily (total of 4 stick packs daily). Twice daily regimen of the food product should occur within 30 minutes

(i.e. 30±5 minutes) before meals (e.g., before breakfast and dinner or before lunch and dinner, if breakfast is not a usual part of their daily routine). If tolerated, subjects will increase administration of the food product to 2 stick packs twice a day (4 stick packs daily) on Days 4 through the Visit 2 (Week 1) visit. At Visit 2, and if tolerated, subject will increase the food product to 3 stick packs twice a day (6 stick packs daily) starting on Day 8 for the remainder of the study.

### Visit 2 (Week 1) to Visit 6 (Week 13):

By the end of the first week, subjects should be administering three stick packs of the composition twice a day (e.g., six stick packs daily) and be maintaining this amount and regimen through Week 13.

### PART 2: Optional Extension Period (12 Weeks) for all Subjects

### Visit 6 (Week 13) to Visit 10 (Week 25):

After the completion of the mandatory 13-week period (Visit 6), subjects from Part 1 have the option to enter Part 2 where subjects (including those that received placebo in Part 1) will receive up to a maximum of 12 additional weeks of the composition. Subjects who do not participate in Part 2 will undergo an end-of-study follow up visit approximately 2-weeks after Visit 6.

Subjects who participate in Part 2 will be provided a supply of the composition at Visit 6 and will be instructed to start consuming 3 stick packs twice daily (6 stick packs daily). There will be at least 4 study visits in Part 2, and all procedures during those visits will be performed. Safety and tolerability of the composition will continue to be assessed as was in Part 1. If subjects drop out at any time for any reason during the study, their last visit should capture all the assessments as the end-of-study assessments.

### SUMMARY OF EXAMPLES

Liver diseases are complex and driven by a multitude of intersecting pathways. Maintaining liver health and function requires coordination of many biological, cellular and molecular processes. As shown in the Examples herein, the amino acid compositions disclosed in this application (e.g. compositions comprising LIRNacCar or LRQNacCar, such as LIRQNacCarS) are able to reduce hepatocyte lipid and inflammation, reduce hepatic stellate cell activation and expression of fibrogenic markers, and modulate macrophages to a less inflammatory, more anti-inflammatory phenotype, whereas compositions tested such as NacCarS and others are only able to influence some, but not all of the important pathways required for maintaining liver health.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition comprising:
a) a leucine amino acid entity;
b) a arginine amino acid entity;
c) glutamine amino acid entity;
d) a N-acetylcysteine (NAC) entity; and
e) one or both of serine amino acid entity or a carnitine entity,
wherein the total wt. % of (a)-(e) is greater than the total wt. % of other amino acid entities in the composition (in dry form), and
wherein optionally one or both of:
the wt. % of the serine amino acid entity is at least 32 wt. % of the amino acid entity components or total components in the composition; or
the wt. % of the carnitine entity is at least 2 wt. % of the amino acid entity components or total components in the composition.

2. The composition of claim 1, wherein the composition further comprises: (f) an isoleucine amino acid entity.

3. The composition of claim 1, wherein the composition does not comprise a peptide of more than 20 amino acid residues in length, or if a peptide of more than 20 amino acid residues in length is present, the peptide is present at less than: 10 weight (wt.) % of the total wt. of the composition (in dry form).

4. The composition of claim 2, wherein one, two, three, four, five, or more of (a)-(f) are in free amino acid form in the composition.

5. The composition of claim 1, wherein the total wt. % of (a)-(e) is greater than the total wt. % of one, two, or three of other amino acid entity components, non-amino acid entity components, or non-protein components in the composition (in dry form).

6. The composition of claim 1, wherein the composition comprises a combination of 18 or fewer amino acid entities.

7. (canceled)

8. The composition of claim 1, wherein one, two, three, or more of methionine, tryptophan, valine, or cysteine is absent from the composition, or if present, are present at less than: 10 wt. % of the total wt. of the composition (in dry form).

9. The composition of claim 1, wherein the composition comprises:
a) the leucine amino acid entity is chosen from:
i) L-leucine or a salt thereof;
ii) a dipeptide or salt thereof, or tripeptide or salt thereof, comprising L-leucine; or
iii) β-hydroxy-β-methylbutyrate (HMB) or a salt thereof;
b) the arginine amino acid entity is chosen from:
i) L-arginine or a salt thereof;
ii) a dipeptide or salt thereof, or tripeptide or salt thereof, comprising L-arginine;
iii) creatine or a salt thereof; or
iv) a dipeptide or salt thereof, or tripeptide or salt thereof, comprising creatine;
c) the glutamine amino acid entity is L-glutamine or a salt thereof or a dipeptide or salt thereof, or tripeptide or salt thereof, comprising L-glutamine;
d) the NAC entity is NAC or a salt thereof or a dipeptide or salt thereof, comprising NAC; and
e) one or both of:
i) the serine amino acid entity is L-serine or a salt thereof or a dipeptide or salt thereof, or tripeptide or salt thereof, comprising L-serine; or
ii) the carnitine entity is L-carnitine or a salt thereof or a dipeptide or salt thereof, or tripeptide or salt thereof, comprising L-carnitine.

10. The composition of claim 9, wherein the composition further comprises: f) L-isoleucine or a salt thereof or a dipeptide or salt thereof, or tripeptide or salt thereof, comprising L-isoleucine.

11. The composition of claim 2, wherein the composition comprises:
a) the leucine amino acid entity is L-leucine or a salt thereof;
b) the arginine amino acid entity is L-arginine or a salt thereof;
c) the glutamine amino acid entity is L-glutamine or a salt thereof;
d) the NAC entity is NAC or a salt thereof;
e) one or both of the serine amino acid entity is L-serine or a salt thereof or the carnitine entity is L-carnitine or a salt thereof; and
f) the isoleucine amino acid entity is L-isoleucine or a salt thereof.

12. The composition of claim 1, present in a unit dosage form comprising 6.7 g+/−20% of amino acid entities.

13. The composition of claim 1, wherein the composition is formulated with a pharmaceutically acceptable carrier.

14-20. (canceled)

21. A method for treating a liver disease or disorder, comprising administering to a subject in need thereof an effective amount of a composition comprising:
a) a leucine amino acid entity;
b) a arginine amino acid entity;
c) glutamine amino acid entity;
d) a N-acetylcysteine (NAC) entity; and
e) one or both of serine amino acid entity or a carnitine entity,
wherein the total wt. % of (a)-(e) is greater than the total wt. % of other amino acid entities in the composition (in dry form); and
wherein optionally one or both of:
the wt. % of the serine amino acid entity is at least 32 wt. % of the amino acid entity components or total components in the composition; or
the wt. % of the carnitine entity is at least 2 wt. % of the amino acid entity components or total components in the composition,
thereby treating the liver disease or disorder in the subject.

22. The method of claim 21, wherein the subject has a fatty liver disease or disorder.

23-26. (canceled)

27. A method of manufacturing a dry blended preparation or PGDBP, comprising at least 5 pharmaceutical grade amino acid entities, said method comprising:
forming a combination of at least 4 pharmaceutical grade amino acid entities and blending the combination for a time sufficient to achieve a dry blended preparation,
wherein the dry blended preparation comprises:
a) a leucine amino acid entity;
b) a arginine amino acid entity;
c) glutamine amino acid entity;
d) a N-acetylcysteine (NAC) entity; and
e) one or both of serine amino acid entity or a carnitine entity.

28. The method of claim 27, wherein the dry blended preparation further comprises (f) an isoleucine amino acid entity.

29. The method of claim 27, wherein:
(i) blending occurs at a temperature lower than 40° C.;
(ii) blending comprises blending or mixing in a blender or mixer at a speed of less than 1000 rpm; or
(iii) the method further comprises one, two, or three of direct blending, roller compaction, or wet granulation of the dry blended preparation.

30. A composition comprising:
a) a leucine amino acid entity;
b) an isoleucine amino acid entity;
c) a arginine amino acid entity;
d) a N-acetylcysteine (NAC) entity; and
e) a carnitine entity,
wherein the total wt. % of (a)-(e) is greater than the total wt. % of other amino acid entities in the composition (in dry form), and
wherein optionally the wt. % of the carnitine entity is at least 2 wt. % of the amino acid entites or the total components in the composition (in dry form).

31. The composition of claim 30, wherein the composition further comprises: (f) one or both of a glutamine amino acid entity or a serine amino acid entity.

32. (canceled)

33. A method for treating a liver disease or disorder, comprising administering to a subject in need thereof an effective amount of a composition comprising:
a) a leucine amino acid entity;
b) an isoleucine amino acid entity;
c) a arginine amino acid entity;
d) a N-acetylcysteine (NAC) entity; and
e) a carnitine entity,
wherein the total wt. % of (a)-(e) is greater than the total wt. % of other amino acid entities in the composition (in dry form), and
wherein optionally the wt. % of the carnitine entity is at least 2 wt. % of the amino acid entities or the total components in the composition (in dry form),
thereby treating the liver disease or disorder in the subject.

34. The composition of claim 30, wherein the composition comprises:
a) the leucine amino acid entity is chosen from:
i) L-leucine or a salt thereof;
ii) a dipeptide or salt thereof, or tripeptide or salt thereof, comprising L-leucine; or
iii) β-hydroxy-β-methylbutyrate (HMB) or a salt thereof;
b) the isoleucine amino acid entity is chosen from:
i) L-isoleucine or a salt thereof;
ii) a dipeptide or salt thereof, or tripeptide or salt thereof, comprising L-isoleucine;
c) the arginine amino acid entity is chosen from:
i) L-arginine or a salt thereof;
ii) a dipeptide or salt thereof, or tripeptide or salt thereof, comprising L-arginine;
iii) creatine or a salt thereof; or
iv) a dipeptide or salt thereof, or tripeptide or salt thereof, comprising creatine;
d) the NAC entity is NAC or a salt thereof or a dipeptide or salt thereof, comprising NAC; and
e) the carnitine entity is L-carnitine or a salt thereof or a dipeptide or salt thereof,
or tripeptide or salt thereof, comprising L-carnitine.

35. The composition of claim 31, wherein the composition comprises:
a) the leucine amino acid entity is L-leucine or a salt thereof;
b) the isoleucine amino acid entity is L-isoleucine or a salt thereof;
c) the arginine amino acid entity is L-arginine or a salt thereof;
d) the NAC entity is NAC or a salt thereof;
e) the carnitine entity is L-carnitine or a salt thereof; and
f) one or both of the glutamine amino acid entity is L-glutamine or a salt thereof or the serine amino acid entity is L-serine or a salt thereof.

36. The composition of claim 30, wherein the composition does not comprise a peptide of more than 20 amino acid residues in length, or if a peptide of more than 20 amino acid residues in length is present, the peptide is present at less than: 10 weight (wt.) % of the total wt. of the composition (in dry form).

37. The composition of claim 31, wherein one, two, three, four, five, or more of (a)-(f) are in free amino acid form in the composition.

38. The composition of claim 30, wherein the composition comprises a combination of 18 or fewer amino acid entities.

39. The composition of claim 30, wherein one, two, three, or more of methionine, tryptophan, valine, or cysteine is absent from the composition, or if present, are present at less than: 10 wt. % of the total wt. of the composition (in dry form).

40. The composition of claim 30, wherein the composition is formulated with a pharmaceutically acceptable carrier.
